(19) United States
(12) Patent Application Publication
(10) Pub. No.: US 2025/0161299 A1
(43) Pub. Date: May 22, 2025

(54) ORAL PCLX-001 IN THE TREATMENT OF HUMAN CANCER

(71) Applicant: PACYLEX PHARMACEUTICALS INC., Calgary (CA)

(72) Inventors: John Robert MACKEY, Edmonton (CA); Neal Mayanrd DAVIES, Edmonton (CA)

(21) Appl. No.: 18/841,472

(22) PCT Filed: Mar. 3, 2023
(86) PCT No.: PCT/CA2023/050281

§ 371 Date: Aug. 26, 2024

Related U.S. Application Data
(60) Provisional application No. 63/316,153, filed on Mar. 3, 2022.

(51) Int. Cl. A61K 31/496; A61K 9/00; A61P 35/00; A61P 35/02

(52) U.S. Cl. CPC A61K 31/496; A61K 9/0053; A61P 35/00; A61P 35/02

(57) ABSTRACT

In one aspect there is provided a method for treating cancer in a subject in need thereof, comprising, administering an oral dosage form of PCLX-001, wherein said oral dosage comprises 20 mg PCLX-001 per day, 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 mg PCLX-001 per day.

### FIELD

The present disclosure relates generally to oral PCLX-001 in the treatment of human cancer.

### BACKGROUND

Myristoylation, the N-terminal modification of proteins with the fatty acid myristate, is critical for membrane targeting of several hundred human proteins [1], including ones critical for intracellular signaling. Because cancer cells often have increased N-myristoyltransferase (NMT) protein expression, NMTs have been proposed as anti-cancer targets [2], but have never previously been studied in human clinical trials.

### SUMMARY

In one aspect there is provided a method for treating cancer in a subject in need thereof, comprising, administering an oral dosage form of PCLX-001, wherein said oral dosage comprises 20 mg PCLX-001 per day, 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 mg PCLX-001 per day.

In one example, said oral dosage provides an effect comprising a T1/2 of about 5 hours to about 18 hours.

In one example, said oral dosage provides an effect comprising a T1/2 of about 5.5 hours to about 14 hours.

In one aspect there is provided a method for treating cancer in a subject in need thereof, comprising, administering an oral dosage form of PCLX-001, wherein said oral dosage comprises 20 mg PCLX-001 per day.

In one example, said oral dosage provides an effect comprising a T1/2 of about 6 hours to about 9 hours.

In one example, said oral dosage provides an effect comprising a T1/2 of about 6.9 hours to about to about 8.9 hours.

In one aspect there is provided a method for treating cancer in a subject in need thereof, comprising, administering an oral dosage form of PCLX-001, wherein said oral dosage comprises 40 mg PCLX-001 per day.

In one example, said oral dosage provides an effect comprising a T1/2 of about 5 hours to about 11 hours.

In one example, said oral dosage provides an effect comprising a T1/2 of about 5.5 hours to about 10.5 hours.

In one aspect there is provided a method for treating cancer in a subject in need thereof, comprising, administering an oral dosage form of PCLX-001, wherein said oral dosage comprises 70 mg PCLX-001 per day.

In one example, said oral dosage provides an effect comprising a T1/2 of about 9 hours.

In one example, said oral dosage provides an effect comprising a T1/2 of about 9.4 hours.

In one aspect there is provided a method for treating cancer in a subject in need thereof, comprising, administering an oral dosage form of PCLX-001, wherein said oral dosage comprises 100 mg PCLX-001 per day.

In one example, said oral dosage provides an effect comprising a T1/2 of about 7 hours to about 8 hours.

In one example, said oral dosage provides an effect comprising a T1/2 of about 7.2 hours to about 7.5 hours.

In one aspect there is provided a method for treating cancer in a subject in need thereof, comprising, administering an oral dosage form of PCLX-001, wherein said oral dosage comprises 140 mg PCLX-001 per day.

In one example, said oral dosage provides an effect comprising a T1/2 of about 6 hours to about 14 hours.

In one example, said oral dosage provides an effect comprising a T1/2 of about 6.7 hours to about 13.8 hours.

In one example, the cancer is Diffuse Large B Cell Lymphoma (DLBCL).

In one example, said DLBC is refractory DLBC.

In one example, the cancer is lymphoma, leukemia, myeloma, breast cancer, small cell lung cancer, non-small cell lung cancer cells; melanoma, adenocarcinoma, pancreas cancer, bladder cancer, ovarian cancer, brain cancer, colon cancer, neuroblastoma, carcinoma, endometrial cancer, sarcoma, thyroid cancer, fibrosarcoma, oral cancer, tongue cancer, nasopharynx cancer, larynx cancer, esophageal cancer, germ cell cancer, stomach cancer, hepatocellular cancer, biliary tract cancer, small intestinal cancer, testis cancer, epidermoid cancer, cervical cancer, kidney cancer, prostate cancer, or pharynx cancer.

In one example, said subject is a human.

In one aspect there is provided a use of an oral dosage form of PCLX-001 for treating cancer in a subject in need thereof, wherein said oral dosage comprises 20 mg PCLX-001 per day, 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 mg PCLX-001 per day.

In one aspect there is provided a use of an oral dosage form of PCLX-001 in the manufacture of a medicament for treating cancer in a subject in need thereof, wherein said oral dosage comprises 20 mg PCLX-001 per day, 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 mg PCLX-001 per day.

In one example, said oral dosage provides an effect comprising a T1/2 of about 5 hours to about 18 hours.

In one example, said oral dosage provides an effect comprising a T1/2 of about 5.5 hours to about 14 hours.

In one aspect there is provided a use of an oral dosage form of PCLX-001 for treating cancer in a subject in need thereof, wherein said oral dosage comprises 20 mg PCLX-001 per day.

In one aspect there is provided a use of an oral dosage form of PCLX-001 in the manufacture of a medicament for treating cancer in a subject in need thereof, wherein said oral dosage comprises 20 mg PCLX-001 per day.

In one example, said oral dosage provides an effect comprising a T1/2 of about 6 hours to about 9 hours.

In one example, said oral dosage provides an effect comprising a T1/2 of about 6.9 hours to about to about 8.9 hours.

In one aspect there is provided a use of an oral dosage form of PCLX-001 for treating cancer in a subject in need thereof, wherein said oral dosage comprises 40 mg PCLX-001 per day.

In one aspect there is provided a use of an oral dosage form of PCLX-001 in the manufacture of a medicament for treating cancer in a subject in need thereof, wherein said oral dosage comprises 40 mg PCLX-001 per day.

In one example, said oral dosage provides an effect comprising a T1/2 of about 5 hours to about 11 hours.

In one example, said oral dosage provides an effect comprising a T1/2 of about 5.5 hours to about 10.5 hours.

In one aspect there is provided a use of an oral dosage form of PCLX-001 for treating cancer in a subject in need thereof, wherein said oral dosage comprises 70 mg PCLX-001 per day.

In one aspect there is provided a use of an oral dosage form of PCLX-001 in the manufacture of a medicament for treating cancer in a subject in need thereof, wherein said oral dosage comprises 70 mg PCLX-001 per day.

In one example, said oral dosage provides an effect comprising a T1/2 of about 9 hours.

In one example, said oral dosage provides an effect comprising a T1/2 of about 9.4 hours.

In one aspect there is provided a use of an oral dosage form of PCLX-001 for treating cancer in a subject in need thereof, wherein said oral dosage comprises 100 mg PCLX-001 per day.

In one aspect there is provided a use of an oral dosage form of PCLX-001 in the manufacture of a medicament for treating cancer in a subject in need thereof, wherein said oral dosage comprises 100 mg PCLX-001 per day.

In one example, said oral dosage provides an effect comprising a T1/2 of about 7 hours to about 8 hours.

In one example, said oral dosage provides an effect comprising a T1/2 of about 7.2 hours to about 7.5 hours.

In one aspect there is provided a use of an oral dosage form of PCLX-001 for treating cancer in a subject in need thereof, wherein said oral dosage comprises 140 mg PCLX-001 per day.

In one aspect there is provided a use of an oral dosage form of PCLX-001 in the manufacture of a medicament for treating cancer in a subject in need thereof, wherein said oral dosage comprises 140 mg PCLX-001 per day.

In one example, said oral dosage provides an effect comprising a T1/2 of about 6 hours to about 14 hours.

In one example, said oral dosage provides an effect comprising a T1/2 of about 6.7 hours to about 13.8 hours.

In one example, the cancer is Diffuse Large B Cell Lymphoma (DLBCL).

In one example, said DLBC is refractory DLBC.

In one example, the cancer is lymphoma, leukemia, myeloma, breast cancer, small cell lung cancer, non-small cell lung cancer cells; melanoma, adenocarcinoma, pancreas cancer, bladder cancer, ovarian cancer, brain cancer, colon cancer, neuroblastoma, carcinoma, endometrial cancer, sarcoma, thyroid cancer, fibrosarcoma, oral cancer, tongue cancer, nasopharynx cancer, larynx cancer, esophageal cancer, germ cell cancer, stomach cancer, hepatocellular cancer, biliary tract cancer, small intestinal cancer, testis cancer, epidermoid cancer, cervical cancer, kidney cancer, prostate cancer, or pharynx cancer.

In one example, said subject is a human.

### BRIEF DESCRIPTION OF THE FIGURES

Embodiments of the present disclosure will now be described, by way of example only, with reference to the attached Figures.

FIG. 1 depicts the chemical structure of PCLX-001 (2,6-dichloro-N-(3-isobutyl-1,5-dimethyl-1H-pyrazol-4-yl)-4-(2-(piperazin-1-yl) pyridin-4-yl)benzene sulfonamide) C₂₄H₃₀C₁₂N₆O₂S.

FIG. 2 depicts computed tomography images of Patient 2 (A) Oct. 29, 2021—TL #2 Segment 5 of Liver, (B) Oct. 29, 2021—TL #2 Segment 5 of Liver (w/o measurements) Image 27/4, (C) Dec. 30, 2021—TL #2 Segment 5 of Liver Image 24/4 with measurements, (D) Dec. 30, 2021—TL #2 Segment 5 of Liver (w/o measurements Image 24/4, (E) Oct. 29, 2021—TL #2 Segment 5 of Liver Image 27/4 (Liver Views), (F) Dec. 30, 2021—TL #2 Segment 5 of Liver Image 24/4 (Liver Views).

FIGS. 3A and 3B depicts non-compartmental analysis for PCLX-001 in Patient 1 (Patient 115-002-001) (A) day 1 (B) day 15.

FIG. 4(A) depicts non-compartmental analysis for PCLX-001 in Patient 2 (Patient 115-002-002) day 1, (B) day 15, (C) depicts non-compartmental analysis for PCLX-001 in Patient 3 (Patient 115-002-004) day 1, (D) day 15.

FIG. 5 depicts nonparametric superposition simulations to steady state in Patient 1 (Patient 115-002-001).

FIG. 6 depicts nonparametric superposition simulations to steady state in Patient 2 (Patient 115-002-002).

FIG. 7 depicts nonparametric superposition simulations to steady state in Patient 3 (Patient 115-002-004).

FIG. 8A and FIG. 8B depict day 1 dose proportionality analysis. FIG. 8A depicts D1 Cmax/Dose vs Dose. FIG. 8B depicts Day 1 AUClast/Dose vs Dose.

FIG. 9A and FIG. 9B depict Day 14 Dose Proportionality Analysis. FIG. 9A depicts Day 14 Cmax/Dose vs Dose. FIG. 9B depicts Day 14 AUClast/Dose vs Dose.

FIG. 10 depicts Steady State Assessment for Subject Not On Pantoprazole Pre-Dose PCLX-001 Concentrations.

FIG. 11A, FIG. 11B, and FIG. 11C, depict Plasma Concentrations of PCLX-001 after Oral Administration of PCLX-001 to Cancer Patients at 20 mg QD.

FIG. 12A, FIG. 12B, and FIG. 12C, depict Plasma Concentrations of PCLX-001 after Oral Administration of PCLX-001 to Cancer Patients at 40 mg QD.

FIG. 13A, FIG. 13B, FIG. 13C, depicts Plasma Concentrations of PCLX-001 after Oral Administration of PCLX-001 to Cancer Patients at 70 mg QD.

FIG. 14A, FIG. 14B, FIG. 14C, FIG. 14D depicts Plasma Concentrations of PCLX-001 after Oral Administration of PCLX-001 to Cancer Patients at 100 mg QD.

FIG. 15A, FIG. 15B, FIG. 15C, depicts Plasma Concentrations of PCLX-001 after Oral Administration of PCLX-001 to Cancer Patients at 140 mg QD.

### DETAILED DESCRIPTION

In one aspect there is provided a method of treating cancer in a subject, comprising: administering an oral dosage form of PCLX-001.

PCLX-001 is also known as DDD86418 (WO 2010/026365)

In one aspect there is provided a method of treating cancer in a subject, comprising: administering an oral dosage form of PCLX-001, wherein said oral dosage comprises 20 mg PCLX-001 per day.

In one aspect there is provided a method of treating cancer in a subject, comprising: administering an oral dosage form of PCLX-001, wherein said oral dosage comprises 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 mg PCLXX-001 per day.

In one aspect there is provided a method of treating cancer in a subject, comprising: administering an oral dosage form of PCLX-001, wherein said oral dosage comprises 20 mg PCLX-001 per day to 420 mg PCLX-001 per day.

In one aspect there is provided a method of treating cancer in a subject, comprising: administering an oral dosage form of PCLX-001, wherein said oral dosage comprises 15 mg PCLX-001 to 500 mg PCLX-001 per day.

The term “cancer”, as used herein, refers to a variety of conditions caused by the abnormal, uncontrolled growth of cells. Cells capable of causing cancer, referred to as “cancer cells”, possess characteristic properties such as uncontrolled proliferation, immortality, metastatic potential, rapid growth and proliferation rate, and/or certain typical morphological features. Cancer cells may be in the form of a tumor, but such cells may also exist alone within a subject, or may be a non-tumorigenic cancer cell.

A cancer may be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer, and detecting a genotype indicative of a cancer. However, a negative result in one or more of the above detection methods does not necessarily indicate the absence of cancer, e.g., a patient who has exhibited a complete response to a cancer treatment may still have a cancer, as evidenced by a subsequent relapse.

It will be appreciated that, in general, determination of the severity of disease requires identification of certain disease characteristics, for example, whether the cancer is pre-metastatic or metastatic, the stage and/or grade of cancer, and the like.

Staging is a process used to describe how advanced a cancer is in a subject. Staging may be important in determining a prognosis, planning treatment and evaluating the results of such treatment. While different cancer staging systems may need to be used for different types of cancer, most staging systems generally involve describing how far the cancer has spread anatomically and attempt to put subjects with similar prognosis and treatment in the same staging group.

Examples of common staging systems used for most solid tumours, some leukemias and lymphomas are the Overall Stage Grouping system and the TMN system. In the Overall Stage Grouping system, Roman numerals I through IV are utilized to denote the four stages of a cancer. Generally, if a cancer is only detectable in the area of the primary lesion without having spread to any lymph nodes it is called Stage I. Stage II and III cancers are generally locally advanced and/or have spread to the local lymph nodes. For example, if the cancer is locally advanced and has spread only to the closest lymph nodes, it is called Stage II. In Stage III, the cancer is locally advanced and has generally spread to the lymph nodes in near proximity to the site of the primary lesion. Cancers that have metastasized from the primary tumour to a distant part of the body, such as the liver, bone, brain or another site, are called, Stage IV, the most advanced stage. Accordingly, stage I cancers are generally small localized cancers that are curable, while stage IV cancers usually represent inoperable or metastatic cancers. As with other staging systems, the prognosis for a given stage and treatment often depends on the type of cancer. For some cancers, classification into four prognostic groups is insufficient and the overall staging is further divided into subgroups. In contrast, some cancers may have fewer than four stage groupings.

A cancer that recurs after all visible tumour has been eradicated is called recurrent disease, with local recurrence occurring in the location of the primary tumour and distant recurrence representing distant metastasis.

Variations to the staging systems may depend on the type of cancer. Moreover, certain types of cancers. The staging system for individual cancers may be revised with new information and subsequently, the resulting stage may change the prognosis and treatment for a specific cancer.

The “grade” of a cancer may be used to describe how closely a tumour resembles normal tissue of its same type. Based on the microscopic appearance of a tumour, pathologists identify the grade of a tumour based on parameters such as cell morphology, cellular organization, and other markers of differentiation. As a general rule, the grade of a tumour corresponds to its rate of growth or aggressiveness and tumours are typically classified from the least aggressive (Grade I) to the most aggressive (Grade IV).

Accordingly, the higher the grade, the more aggressive and faster growing the cancer. Information about tumour grade is useful in planning treatment and predicting prognosis.

In some examples, in the case of lymphoma, Stage 1 refers to lymphoma in only one group of lymph nodes. Stage II refers to two or more groups of lymph nodes are affected but they are all either above or below the diaphragm, either all in the chest or all in the abdomen. Stage III refers to two or more groups of lymph nodes are affected in both the chest and the abdomen. Stage IV refers to lymphoma is in at least one organ (e.g., bone marrow, liver or lungs) as well as the lymph nodes. Additional designations may be added to the foregoing stages. For example, “A” generally means the patient has not experiences any troublesome symptoms. “B” means the patient has experienced B symptoms (e.g., fever, night sweats, weight loss). X means the patient has bulky disease (e.g., large tumour greater than 10 cm in size). E means the patient has extranodal disease (e.g., disease outside the lymph nodes).

In a specific example, the cancer is a lymphoma.

The term “lymphoma” generally refers to a malignant neoplasm of the lymphatic system, including cancer of the lymphatic system. The two main types of lymphoma are Hodgkin's disease (HD or HL) and non-Hodgkin's lymphoma (NHL). Abnormal cells appear as congregations which enlarge the lymph nodes, form solid tumours in the body, or more rarely, like leukemia, circulate in the blood. Hodgkin's disease lymphomas, include nodular lymphocyte predominance Hodgkin's lymphoma; classical Hodgkin's lymphoma; nodular sclerosis Hodgkin's lymphoma; lymphocyterich classical Hodgkin's lymphoma; mixed cellularity Hodgkin's lymphoma; lymphocyte depletion Hodgkin's lymphoma. Non-Hodgkin's lymphomas include small lymphocytic NHL, follicular NHL; mantle cell NHL; mucosa-associated lymphoid tissue (MALT) NHL; diffuse large cell B-cell NHL; mediastinal large B-cell NHL; precursor T lymphoblastic NHL; cutaneous T-cell NHL; T-cell and natural killer cell NHL; mature (peripheral) T-cell NHL; Burkitt's lymphoma; mycosis fungoides; Sezary Syndrome; precursor B-lymphoblastic lymphoma; B-cell small lymphocytic lymphoma; lymphoplasmacytic lymphoma; splenic marginal zone B-cell lymphoma; nodal marginal zone lymphoma; plasma cell myeloma/plasmacytoma; intravascular large B-cell NHL; primary effusion lymphoma; blastic natural killer cell lymphoma; enteropathy-type T-cell lymphoma; hepatosplenic gamma-delta T-cell lymphoma; subcutaneous panniculitis-like T-cell lymphoma; angioimmunoblastic Tcell lymphoma; primary systemic anaplastic large T/null cell lymphoma, diffuse large B-cell lymphoma (DLBCL), refractory diffuse large B-cell lymphoma (DLBCL).

In a specific example, the lymphoma is a B-cell lymphoma.

In one example, the cancer is diffuse large B-cell lymphoma (DLBCL).

In one example, the cancer is refractory diffuse large B-cell lymphoma (DLBCL).

In some examples, the compositions and/or compositions described herein (for example, PCLX-001) may be used to treat various stages and grades of cancer development and progression. In some examples, PCLX-001 may be used in the treatment of early stage cancers including early neoplasias that may be small, slow growing, localized and/or nonaggressive, for example, with the intent of curing the disease or causing regression of the cancer, as well as in the treatment of intermediate stage and in the treatment of late stage cancers including advanced and/or metastatic and/or aggressive neoplasias, for example, to slow the progression of the disease, to reduce metastasis or to increase the survival of the patient. Similarly, PCLX-001 may be used in the treatment of low grade cancers, intermediate grade cancers and or high grade cancers.

In some examples, it is contemplated that PCLX-001 may be used in the treatment of indolent cancers, recurrent cancers including locally recurrent, distantly recurrent and/or refractory cancers (i.e., cancers that have not responded to treatment), metastatic cancers, locally advanced cancers and aggressive cancers.

In another example, the cancer is leukemia, myeloma, breast cancer, small cell lung cancer, non-small cell lung cancer cells; melanoma, adenocarcinoma, pancreas cancer, bladder cancer, ovarian cancer, brain cancer, colon cancer, neuroblastoma, carcinoma, endometrial cancer, sarcoma, thyroid cancer, fibrosarcoma, oral cancer, tongue cancer, nasopharynx cancer, larynx cancer, esophageal cancer, germ cell cancer, stomach cancer, hepatocellular cancer, biliary tract cancer, small intestinal cancer, testis cancer, epidermoid cancer, cervical cancer, kidney cancer, prostate cancer, or pharynx cancer

In some examples, PCLX-001 may be used alone or in combination with one or more therapeutic agents as part of a primary therapy or an adjuvant therapy. “Primary therapy” or “first-line therapy” refers to treatment upon the initial diagnosis of cancer in a subject. Exemplary primary therapies may involve surgery, a wide range of chemotherapies, immunotherapy and/or radiotherapy. When first-line or primary therapy is not systemic chemotherapy or immunotherapy, then subsequent chemotherapy or immunotherapy may be considered as “first-line systemic therapy”. In one example, PCLX-001 may be used for first-line systemic therapy.

The term “adjuvant therapy” refers to a therapy that follows a primary therapy and that is administered to subjects at risk of relapsing. Adjuvant systemic therapy is typically begun soon after primary therapy to delay recurrence, prolong survival or cure a subject. Treatment of a refractory cancer may be termed a “second-line therapy” and is a contemplated use of the present invention, in addition to first-line therapy.

In some examples, a subject is also treated with a proton pump inhibitor (PPI). PPIs are typically prescribed for short-term treatment of active duodenal ulcers, gastrointestinal ulcers, gastroesophageal reflux disease (GERD), severe erosive esophagitis, and/or poorly responsive symptomatic GERD.

Examples of proton pump inhibitors include, omeprazole (Prilosec®), lansoprazole (Prevacid®), esomeprazole (Nexium®), rabeprazole (Aciphex®), pantoprazole (Protonix®), pariprazole, tenatoprazole, and leminoprazole. The drugs of this class suppress gastrointestinal acid secretion by the specific inhibition of the H+/K+-ATPase enzyme system (proton pump) at the secretory surface of the gastrointestinal parietal cell.

In a specific examples, the PPI used is pantoprazole.

In one embodiment of the present invention, the combinations are used in the treatment of an early stage cancer. In another embodiment, the combinations are used as a first-line systemic therapy for an early stage cancer.

In an alternate example, PCLX-001 may be used in the treatment of a late stage and/or advanced and/or metastatic cancer. In a further embodiment, PCLX-001 may be administered as a first-line systemic therapy for the treatment of a late stage and/or advanced and/or metastatic cancer.

A refractory cancer refers to a cancer that is not amenable to surgical intervention and the cancer is either initially unresponsive to chemo- or radiation therapy or the cancer becomes unresponsive over time.

The term “relapse” refers to those patients who achieved a clinical response (CR) or partial response (PR) and subsequently experienced disease progression.

The term “clinical response” refers to inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others.

The term “overall survival” or “OS” refers to the time from enrollment to death from any cause.

The term “duration of response” or “DOR” refers to the time from the first occurrence of a documented objective response to disease progression.

The term “progression free survival” or “PFS” refers to the time from enrollment to the date of the first recorded occurrence of disease progression.

The term “stable disease” or “SD” refers to neither sufficient shrinkage to qualify for CR or PR nor sufficient increase growth of tumor to qualify for PD.

The term “partial response” refers to persistence of one or more non-target lesions and/or (if applicable) maintenance of tumor marker level above the normal limits.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal.

In a specific example, the subject is a human.

The term “treatment” or “treat” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early cancer, for example an early stage lymphoma, can be treated to prevent progression or alternatively a subject in remission can be treated with a compound or composition described herein to prevent recurrence.

The term “pharmaceutically acceptable” as used herein includes compounds, materials, compositions, and/or dosage forms (such as unit dosages) which are suitable for use in contact with the tissues of a subject without excessive toxicity, irritation, allergic response, or other problem or complication, commensurate with a reasonable benefit/risk ratio. Each carrier, excipient, etc. is also be “acceptable” in the sense of being compatible with the other ingredients of the formulation.

The term “excipient” means a pharmacologically inactive component such as a diluent, lubricant, surfactant, carrier, or the like. Excipients that are useful in preparing a pharmaceutical composition are generally safe, non-toxic and are acceptable for human pharmaceutical use. Reference to an excipient includes both one and more than one such excipient.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), stabilizers and preservatives, and the like.

A “treatment or dosage regimen” as used herein refers to a combination of dosage, frequency of administration, or duration of treatment, with or without addition of a second medication.

The term “diagnosis” as used herein, refers to the identification of a molecular and/or pathological state, disease or condition, such as the identification of lymphoma, or other type of cancer.

The term “alleviates” as used herein refers to a decrease, reduction or elimination of a condition, disease, disorder, or phenotype, including an abnormality or symptom.

In some aspects there is provided a pharmaceutical composition comprising PCLX-001 in admixture with a pharmaceutically acceptable adjuvant, diluent or carrier.

Pharmaceutical compositions of the present disclosure may also contain adjuvants such as preservative, wetting agents, emulsifying agents and dispersing agents. Prevention of the action of microorganisms may be ensured by the inclusion of various antibacterial and antifungal agents, for example, paraben, chlorobutanol or phenol sorbic acid. It may also be desirable to include isotonic agents such as sugars or sodium chloride, for example.

Solid dosage forms for oral administration include capsules, tablets, pills, powders and granules. In such solid dosage forms, an active compound (e.g., PCLX-001) is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

The term “solid” as used herein refers to a substance that is solid or semi-solid at room temperature. Hence, as used herein, a “solid” substance may become liquid at, e.g., body temperature.

Cmax is the maximum plasma concentration of PCLX-001.

Tmax is the time at which the maximum plasma concentration of PCLX-001 is achieved.

The term “pharmacokinetic” (or PK) refers to the factors which determine the attainment and maintenance of the appropriate concentration of drug at a site of action.

The term “AUC” refers to the area under concentration (or area under the curve) representing the concentration of a compound or metabolite thereof in a biological fluid of a patient as a function of time following administration of the compound to the patient.

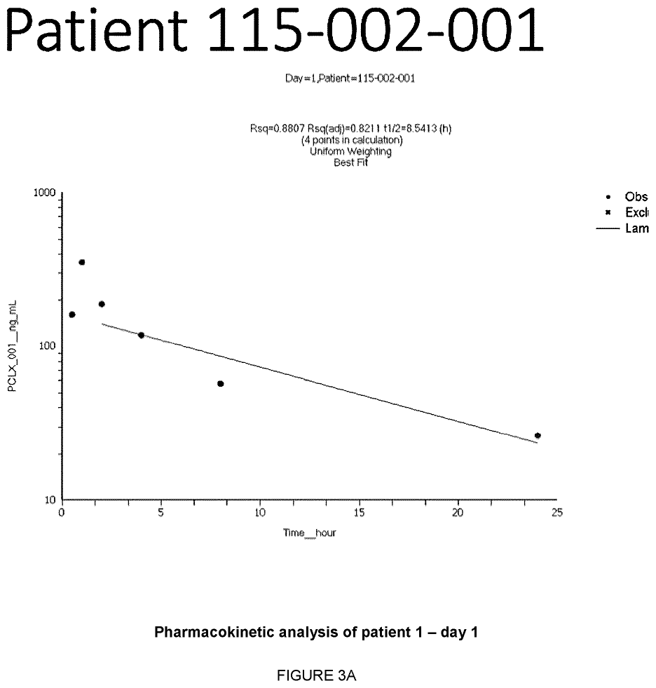

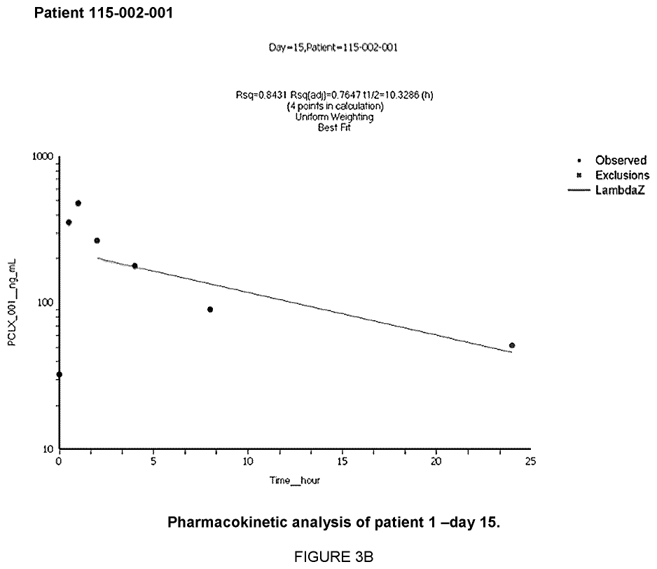

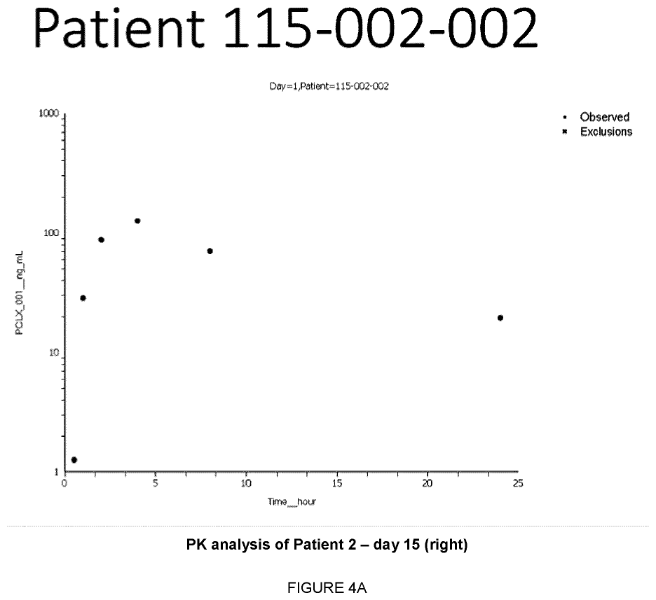

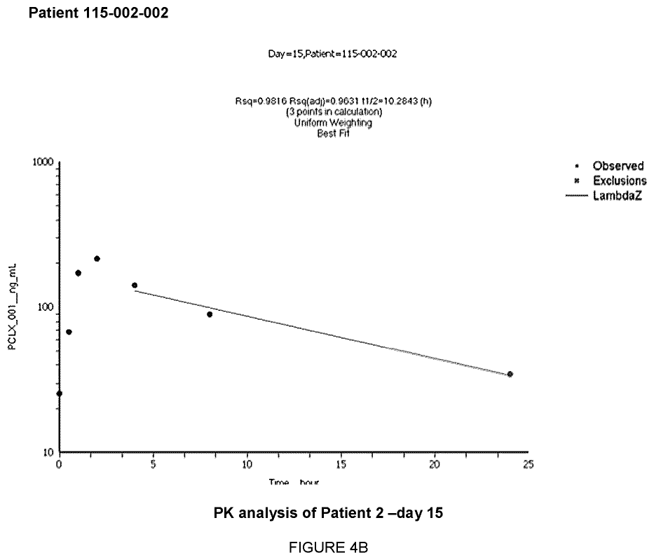

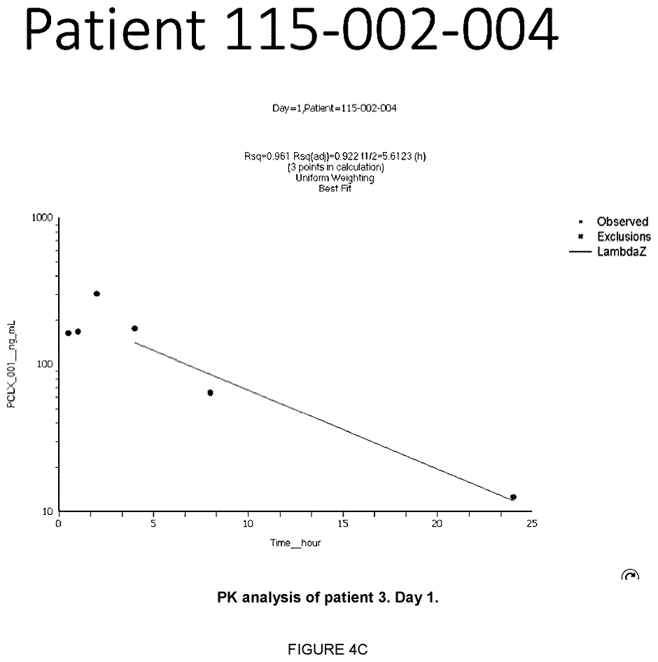

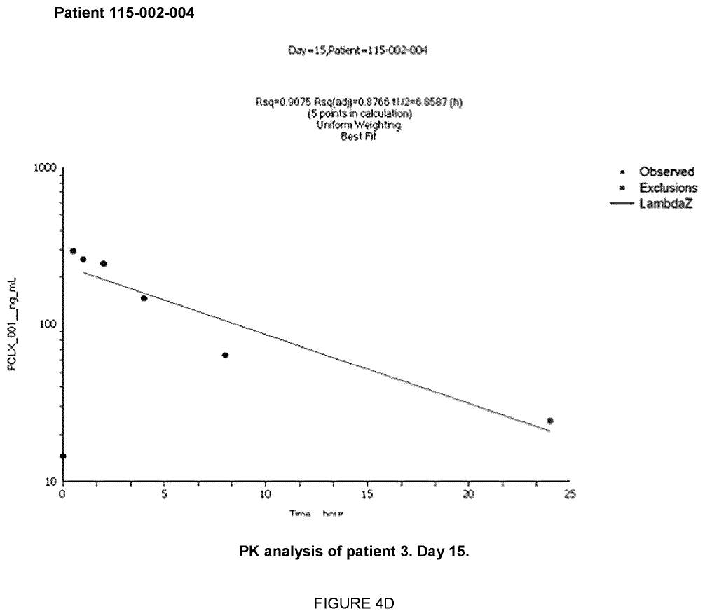

Pharmacokinetic analysis of PCLX-001 is shown herein. Table 1A summarizes the measured pharmacokinetic parameters of Patient 1, Patient 2, and Patient 3, respectively. FIGS. 3, 4A-B, and 4C-D, show the single dose 20 mg oral pharmacokinetic profile of Patient 1, Patient 2, and Patient 3, respectively.

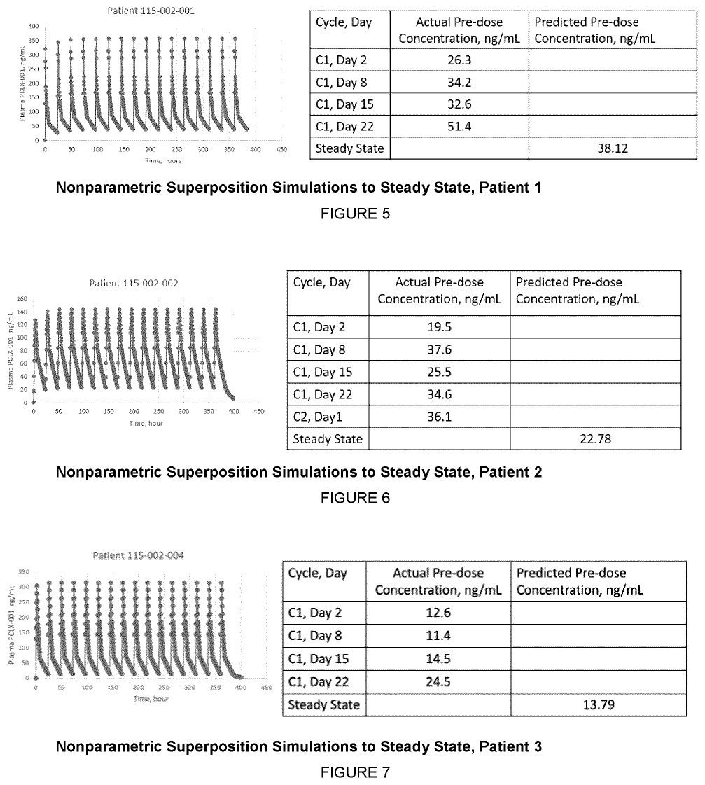

Surprisingly, as shown herein, the trough concentrations (see FIGS. 5, 6, and 7) were nearing the levels we see to treat cell lines ex vivo (Table 2). These findings were unexpected because, in part, allometric scaling from non-human species suggested much higher doses would be required for efficacy (Table 3).

In the example of Patient 2, the efficacy of the dosing regimen and therapeutic utility was determined.

Patient 2 was a fifty-six year old woman with heavily pretreated DLBCL and was the second patient on a phase 1 dose escalation trial of PCLX-001. The first daily oral administration of 20 mg PCLX-001 tablets achieved a lower Cmax and a lower extent of exposure were apparent compared to the patient 1, and while terminal half-life could not be accurately estimated, significant drug exposure was noted. Furthermore day 15 PK analysis suggested a terminal half-life of approximately 10 hours. No significant drug accumulation was noted, and nonparametric superposition suggested steady state concentrations were reached quickly. These characteristics were favorable for once a day dosing and also could support more frequent administration, or a modified release formulation.

In a finding unexpected and unanticipated by allometric scaling of PK results in mice, rats, and dogs, PCLX-001 dosing achieved through concentrations (those measured immediately prior to dosing on day 2, 15, and 22) higher than anticipated in each of patients 1, 2, and 3. Peak plasma concentrations exceeded those needed to suppress cultured human cancer cell growth. Trough plasma concentrations approached those needed to suppress cultured human cancer cell growth. Furthermore, the treatment effect of PCLX-001 in patient 2 was demonstrated by a marked reduction of volume of hepatic metastatic disease on comparison of pre-treatment and post-treatment computed tomographic images.

By allometric scaling from mouse, it was predicted approximately 180 mg po qd would be needed to achieve therapeutic results. However, as shown herein, signs of efficacy are shown at 20 mg.

The allometric scaling is based on toxicity, and the toxicity is that which establishes the Maximally Tolerated Dose (MTD) in animal studies. In mice, one toxicity study established the MTD to be 35 mg/kg. In other studies with mice, 35 mg/kg and higher doses had anti-cancer effects in mice with implanted human tumors (3). Dosing mice bearing human cancers at 10 mg/kg or lower had, in general, no evident anticancer activity. It was not expected that, as shown herein, doses markedly lower than 180 mg daily in humans would have anticancer effects.

In one example, there is provided a method for treating cancer in a subject in need thereof, comprising, administering an oral dosage form of PCLX-001, wherein said oral dosage comprises 20 mg PCLX-001 per day.

In another example, there is provided a method for treating cancer in a subject in need thereof, comprising, administering an oral dosage form of PCLX-001, wherein said oral dosage comprises 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 mg PCLX-001 per day.

In one example, wherein when said oral dosage comprises 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 mg PCLX-001 per day, said oral dosage provides an effect comprising a T1/2 of 8±3 h. In another example, said oral dosage provides an effect comprising a T1/2 of 8 h.

In one example, wherein when said oral dosage comprises 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 PCLX-001 per day said oral dosage provides an effect comprising a Tmax of about 2.25±1.75 hours. In another example, said oral dosage provides an effect comprising a Tmax of about 2.25 hours.

In vitro studies of PCLX-001 have shown inhibition of cell growth of hematological cancer cells, including lymphomas, leukemia, and myeloma; breast cancer cells; small cell lung cancer cells; non-small cell lung cancer cells; melanoma cells; adenocarcinoma cells; pancreas cancer cells; bladder cancer cells; ovary cancer cells; brain cancer cells; colon cancer cells; neuroblastoma; carcinoma cancer cells; endometrial cancer cells; sarcoma cancer cells; thyroid cancer cells; Fibrosarcoma cancer cells; tongue cancer cells; testis cancer cells; epidermoid cancer cells; cervix cancer cells; kidney cancer cells; prostate cancer cells; and pharynx cancer cells. (3)

The term “bioavailability” refers to the rate and amount of a drug that reaches the systemic circulation of a patient following administration of the drug or prodrug thereof to the patient and can be determined by evaluating, for example, the plasma or blood concentration-versus-time profile for a drug. Parameters useful in characterizing a plasma or blood concentration-versus-time curve include the area under concentration (AUC), the time to peak concentration (Tmax), and the maximum drug concentration (Cmax)

Absolute oral bioavailability is the bioavailability of a compound or metabolite thereof following oral administration compared to the bioavailability following intravenous administration of an equivalent amount of the compound or metabolite thereof. Relative oral bioavailability of a compound or metabolite thereof is the bioavailability following oral administration of a compound or metabolite thereof relative to administration of an equivalent amount of the compound or metabolite thereof in another dosage form and/or route of administration.

The term “Dosage form” refers to a form of a formulation that contains an amount of active agent or prodrug of an active agent, e.g., PCLX-001, which may be administered to a patient to achieve a therapeutic effect.

An oral dosage form is intended to be administered to a patient taken orally, for example via the mouth and swallowed.

The terms “taken orally” and “oral administration” refer to a manner of providing an active agent to a subject or patient by the mouth through the gastrointestinal tract (digestive tract, digestional tract, GI tract, GIT, gut, or alimentary canal) and are used interchangeably. The gastrointestinal tract is an organ system within humans and other animals which takes in food, digests it to extract and absorb energy and nutrients, and expels the remaining waste as feces. The mouth, esophagus, stomach and intestines are part of the gastrointestinal tract.

Suitably, oral formulations may contain a dissolution aid. The dissolution aid is not limited as to its identity so long as it is pharmaceutically acceptable. Examples include non-ionic surface active agents, such as sucrose fatty acid esters, glycerol fatty acid esters, sorbitan fatty acid esters (e.g. sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g. chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions include polymeric substances and waxes.

PCLX-001 may also be in a micro-encapsulated form, if appropriate, with one or more of the above-mentioned excipients.

Liquid dosage forms for oral administration include pharmaceutically acceptable emulsions, solutions, suspensions, syrups and elixirs. In addition to PCLX-001, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

In other examples, pharmaceutical compositions of the present disclosure may be used for parenteral injection suitably comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

Prolonged absorption of an injectable pharmaceutical form may be brought about by the inclusion of agents (for example aluminum monostearate and gelatin) which delay absorption.

In some cases, in order to prolong the effect of the pharmaceutical compositions of the present disclosure, it is desirable to slow the absorption of the composition, or PCLX-001 from subcutaneous or intramuscular injection. This may be accomplished by the use of a liquid suspension, where PCLX-001 is suspended in. The rate of absorption of PCLX-001 then depends upon its rate of dissolution.

Alternatively, delayed absorption of a parenterally administered pharmaceutical composition of the present disclosure is accomplished by dissolving or suspending PCLX-001 in an oil vehicle. Injectable depot forms are suitably made by forming microencapsulated matrices of PCLX-001 in biodegradable polymers, for example polylactide-polyglycolide. Depending upon the ratio of PCLX-001 to polymer and the nature of the particular polymer employed, the rate of release can be controlled. Examples of other biodegradable polymers include poly(orthoesters) and poly(anhydrides).

Depot injectable formulations may also be prepared by entrapping PCLX-001 in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

In other examples, pharmaceutical compositions of the present disclosure for rectal or vaginal administration are preferably suppositories which can be prepared by mixing PCLX-001 with suitable non-irritating excipients or carriers such as cocoa butter, polyethylene glycol or a suppository wax which are solid at room temperature but liquid at body temperature and therefore melt in the rectum or vaginal cavity and release PCLX-001.

PCLX-001 may also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. Pharmaceutical compositions of the present disclosure in liposome form can contain, in addition to PCLX-001, stabilisers, preservatives, excipients and the like. The preferred lipids are the phospholipids and the phosphatidyl cholines (lecithins), both natural and synthetic. Methods to form liposomes are known in the art.

Dosage forms for topical administration of PCLX-001 include powders, sprays, ointments and inhalants. PCLX-001 may be mixed under sterile conditions with a pharmaceutically acceptable carrier and any needed preservatives, buffers or propellants which may be required. PCLX-001 may also be administered, for example as part of a pharmaceutical composition, in ophthalmic formulations, or eye ointments, powders and solutions.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Abstract

Many human cancers are inadequately treated with existing anti-cancer drugs. Among such cancers, relapsed and refractory Diffuse Large B Cell Lymphoma (DLBCL) requires new therapeutic approaches, particularly for transplant/CAR-T ineligible and elderly patients. We describe two patients with DLBCL treated with a novel anti-cancer drug, and a third patient with heavily pretreated soft tissue sarcoma. Patient 1 was an eighty-six year old woman with heavily pretreated DLBCL who received a novel, first in class small molecule inhibitor of N-myristoyltransferase (NMT) as the initial patient on a phase I dose escalation trial. Daily oral administration of 20 mg PCLX-001 tablets produced a pharmacokinetic profile suitable for single daily dosing: rapid oral absorption followed by an apparent elimination half-life of 8.54 hours on day 1 and 10.3 hours on day 15, a substantial trough concentration of drug, and without systemic accumulation of drug by day 15. The patient did not experience any dose limiting toxicities but had disease progression after twenty-eight days of study therapy. Patient 2 was a fifty-six year old woman with heavily pretreated DLBCL and was the second patient on the phase 1 dose escalation trial of PCLX-001. Daily oral administration of 20 mg PCLX-001 tablets achieved a lower Cmax and longer Tmax and a lower extent of exposure were apparent compared to the patient 1. In a finding unexpected and unanticipated by allometric scaling of PK results in mice, rats, and dogs, PCLX-001 dosing achieved through concentrations (those measured immediately prior to dosing on day 2, 15, and 22) higher than anticipated in each of patients 1, 2, and 3. Peak plasma concentrations exceeded those needed to suppress cultured human cancer cell growth. Trough plasma concentrations approached those needed to suppress cultured human cancer cell growth. Furthermore, the treatment effect of PCLX-001 in patient 2 was demonstrated by a marked reduction of volume of hepatic metastatic disease on comparison of pre-treatment and post-treatment computed tomographic images. Patient 3 was a 55 year old woman with heavily pretreated soft tissue sarcoma and was the third patient on the phase 1 dose escalation trial of PCLX-001. Daily oral administration of 20 mg PCLX-001 tablets had PK characteristics consistent with the requirements for daily oral administration. Estimated half-life was 5.61 h on day 1 and 6.86 h on day 15. Peak plasma concentrations exceeded those needed to suppress cultured human cancer cell growth. Trough plasma concentrations approached those needed to suppress cultured human cancer cell growth.

### INTRODUCTION

PCLX-001 is a potent, small molecule inhibitor of both human NMT proteins, NMT1 and NMT2. Preclinical studies showed PCLX-001 markedly inhibited hematologic and lymphoma cell lines in tissue culture, achieved complete remissions in human cancers grown in immunodeficient mice [3] and tumour responses in solid cancers [4]. Although PCLX-001 has multiple potential mechanisms of action, in B-cell lymphoma models it inhibits early B-cell receptor (BCR) signaling events critical for survival. In addition to abrogating myristoylation of Src family kinases, PCLX-001 also promotes their degradation and that of numerous non-myristoylated BCR effectors including c-Myc, NFκB and P-ERK, leading to cancer cell death in vitro and in xenograft models [3]. The molecule has been extensively investigated in non-clinical safety testing [5], and found suitable for formal drug development in humans.

Herein, we report the first three patients treated with an NMT inhibitor. Pharmacokinetic endpoints were explored in this first-in-human drug exposure, showing PCLX-001 has pharmacokinetic properties suitable for an oral, once daily, cancer therapy.

### 2. Materials and Methods

### 2.1. PCLX-001—the Investigational Agent

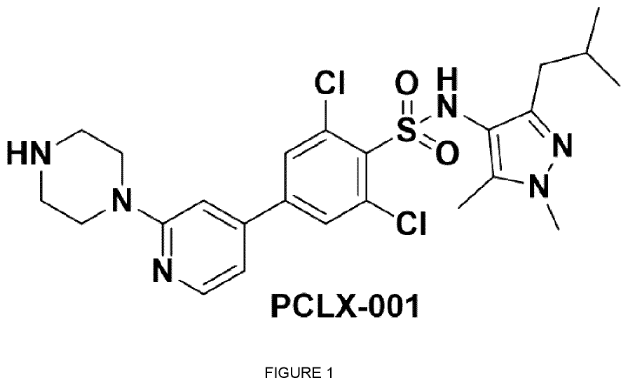

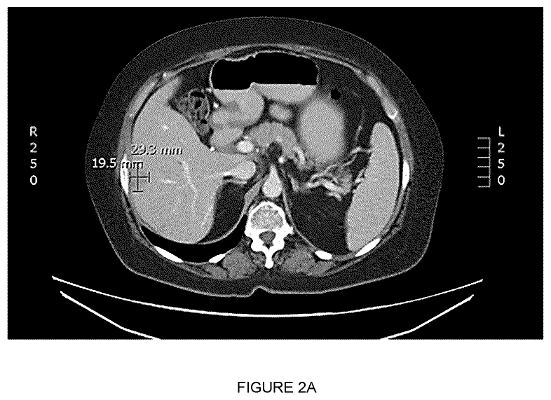

PCLX-001 (FIG. 1) is a potent, small molecule inhibitor of human NMT1 and NMT2 proteins. In animal models, it has complete oral bioavailability. There is no significant off target kinase inhibition, as demonstrated by a KINOMEscan (Fremont, CA, USA) [3]. Preclinical testing reveals no significant hERG interaction and animal non-clinical safety testing showed no cardiac conduction issues. In GLP non-clinical safety testing, diarrhea was dose limiting [5]. Mechanism of action studies show that while NMT inhibition can affect approximately six hundred human proteoforms [1], in models of human lymphoma Bruton tyrosine kinase (BTK) modification and downstream signaling was profoundly inhibited in the presence of PCLX-001 [3].

### 2.2. Clinical Trial

The clinical trial is entitled “Phase I Trial of PCLX-001 in B-cell Non-Hodgkin Lymphoma and Advanced Solid Malignancies” and registered at clinicaltrials.gov as NCT04836195. It is a phase I dose-escalation study of oral PCLX-001, conducted in a multicenter, non-randomized, open-label, non-controlled design. The study is comprised of two parts: Part A (single-agent dose escalation) and Part B (single-agent expansion cohorts) [6].

The primary endpoint of the study is to determine, during the dose escalation phase, the recommended dose of PCLX-001 for the dose expansion phase of the trial.

Patients are to receive daily oral PCLX-001 on 28-day cycles, beginning with a starting dose of 20 mg.

The study was conducted according to the guidelines of the Declaration of Helsinki, and approved by the Institutional Review Board of the Cross Cancer Institute (postal code T6G1Z2 and protocol code HREBA CC-21-0157, approved Jun. 24, 2021). Informed consent was obtained from all subjects involved in the study.

### 2.3. Pharmacokinetic Analysis

The primary pharmacokinetic analysis endpoints were
i) To determine the time to maximum plasma level (Tmax) of PCLX-001 measured during Cycle 1: Pre-dose on Days 1, 2, 8 (+2), 15 (+2), and 22 (+2); Day 1 and Day 15 will also be measured post-dose at 0.5, 1, 2, 4, and 8 hours; Cycle 2 pre-dose on Day 1.
Tmax is the time at which the maximum plasma concentration of PCLX-001 is achieved.
ii) To determine the maximum plasma level (Cmax) of PCLX-001 measured on Cycle 1: Pre-dose on Days 1, 2, 8 (+2), 15 (+2), and 22 (+2); Day 1 and Day 15 will also be measured post-dose at 0.5, 1, 2, 4, and 8 hours; Cycle 2 pre-dose on Day 1.
Cmax is the maximum plasma concentration of PCLX-001.

The patient was dosed with oral doses of PCLX-001 20 mg. After dosing, the blood samples (volumes 3 mL) were serially collected into heparinized tubes from a right forearm butterfly cannula. All blood fluid specimens were frozen and later assayed using a validated UHPLC-MS detection method.

Noncompartmental pharmacokinetic analysis was performed using Phoenix WinNonlin 8.3 using trapezoidal rule and linear up log down methodology. WinNonlin was allowed to pick optimal terminal phases. Results are shown in Table 1A.

Nonparametric superposition was performed using Phoenix WinNonlin Nonparametric Superposition to predict steady state data based upon Day 1 data for each patient. It utilized the 8- and 24-hour time points for estimation of terminal phase for the purposes of this exercise. We simulated 20 mg daily oral dosing and simulations were conducted out to steady state. Results are show in FIGS. 5, 6, and 7.

### 2.4. Toxicity Assessment

The subjects were assessed for toxicity using daily diary entries, clinical assessments at baseline, day 8, day 15, day 22, and day 28. Dose limiting toxicities were DLTs defined as the following cycle 1 CTCAE version 5.0 adverse events: Gr 4 platelets, Gr≥3 platelets with bleeding and/or transfusions, Gr 4 ANC for ≥7 days, Gr≥3 febrile neutropenia, and Gr≥3 non-hematological toxicity.

### 2.5. Efficacy Assessment

Patients 1 and 2 had baseline cross sectional and PET imaging, and patient 3 had baseline cross sectional imaging. Protocol defined imaging was CT every 2 cycles and PET every 6 cycles or as clinically indicated. Evaluation of response was by the Lugano classification criteria for the evaluation of non-Hodgkin lymphoma, and RECIST 2.0 for the evaluation of soft tumors.

### 3. Results-Patient 1

### 3.1. Patient Demographics

The patient (Patient 1) was an 86 year-old Caucasian woman with refractory, relapsed diffuse large B-cell lymphoma (DLBCL), with immunohistochemical subtyping consistent with germinal center B-cell (GCB) as the cell of origin. Comorbidities included hypothyroidism, hypertension, osteoporosis, and endovascular repair of abdominal aortic aneurysm in 2018. The original cancer diagnosis was Stage IVA DLCBCL, non-GCB cell of origin (CD10 negative, BCL2, BCL6, MUM1 positive) in September 2014, for which she received R-CHOP (rituximab, cyclophosphamide, doxorubicin, vincristine, prednisone) chemotherapy. The relapse was diagnosed July of 2021 as Stage IVA DLBCL, GCB cell of origin (CD10 negative, MUM1 negative, BCL2, BCL6 positive), for which she received ibrutinib. Immediately prior to study entry, baseline bloodwork showed normal hematopoiesis and biochemistry. Computed tomography demonstrated a lingular mass, cervical, hilar, mediastinal nodal involvement, and T10 infiltration. Bone marrow biopsy of the iliac crest was non-diagnostic.

### 3.2. PCLX-001 Administration

PCLX-001 was administered orally each morning as two tablets, each containing PCLX-001 10 mg. PCLX-001 was initiated on Sep. 14, 2021, and discontinued on day 29 due to symptomatic back pain with CT confirming progression of lymph nodes.

### 3.3. Pharmacokinetics

The pharmacokinetic analysis was conducted as described above. FIG. 3 shows the plasma concentration of PCLX-001 after the initial oral dose and after day 15 dosing. Table 1A shows the pharmacokinetic parameters determined by this analysis. Day 1 results show a rapid absorption rate, with peak plasma concentration achieved at 1 hours after oral administration, a peak plasma concentration of 353 ng/ml, and an apparent terminal half-life of 8.54 hours. Day 15 results show rapid absorption with a peak plasma concentration of 479 ng/ml achieved at 1 hour after administration. Nonparametric superposition simulations (FIG. 7) show rapid achievement of steady state kinetics. These parameters are all supportive of a single daily oral dosing schedule for PCLX-001, although they could support more frequent oral dosing or the use of a prolonged release formulation.

TABLE 1A
Pharmacokinetic analysis results of patient 1, 2, and 3.

| | | | | | | AUCₗₐₛₜ or | | |
|---|---|---|---|---|---|---|---|---|
| | | Dose | Half-life | Tₘₐₓ | Cₘₐₓ | AUCₜₐᵤ, | AUC_{inf} | AUCₑₓₜᵣₑₚ |
| Day | Patient | (mg) | (h) | (h) | (ng/mL) | (ng*h/mL) | (ng*h/mL) | (%) |
| 1 | 115-002-001 | 20 | 8.54* | 1 | 353 | 1710 | 2034* | 15.9 |
| 1 | 115-002-002 | 20 | | 4 | 127 | 1305 | | |
| 1 | 115-002-004 | 20 | 5.61 | 2 | 304 | 1783 | 1885 | 5.41 |
| 15 | 115-002-001 | 20 | 10.3* | 1 | 479 | 2732 | 3498* | 21.9 |
| 15 | 115-002-002 | 20 | 10.3* | 2 | 215 | 2008 | 2521* | 20.4 |
| 15 | 115-002-004 | 20 | 6.86* | 0.5 | 295 | 1912 | 2154** | 11.3 |

*WinNonlin fit does not describe terminal phase well.

For data that are underlined, the terminal half-life and AUCinf could not be adequately characterized.

A different pharmacokinetic modeling program was used in the publication PK Solver 2.0 freeware (https://www.boomer.org/boomer/software/pksolver.zip; accessed 26 Nov. 2021) was used to determine the single dose pharmacokinetic parameters using a two-compartment model. This used a compartmental modeling approach vs the patent application which does not assuming compartments and models without them letting data speak for itself without constraints of a compartment model. As noted above, non-compartmental pharmacokinetic analysis was performed using Phoenix WinNonlin 8.3 using trapezoidal rule and linear up log down methodology. WinNonlin was allowed to pick optimal terminal phases.

The results are shown in Table 1B.

Pharmacokinetic analysis was conducted as described above. Table 1B shows the pharmacokinetic parameters determined by this analysis using two different software and compartmental vs non-compartmental methods. These parameters are all supportive of a single daily oral dosing schedule for PCLX-001.

TABLE 1B
Comparing two different methods used and two
different pharmacokinetic modeling programs.

| | | | | | Auc•last• | | AUC• |
|---|---|---|---|---|---|---|---|
| | | Dose• | Half- | Cmax• | or | AUC•inf | extrap• |
| Day | Patient | (mg) | Life•(h) | (ng/mL) | (ng*h/mL) | (ng*h/mL) | (%) |
| 1 | 115-002-001 | 20 | 8.54 | 353 | 1710 | 2034 | 15.9 |
| 15 | 115-002-001 | 20 | 10.3 | 479 | 2732 | 3498 | 21.9 |
| 15 | 115-002-001 | 20 | 16.42 | 278.23 | 1701.33 | 2355.81 | NR |
| 15 | 115-002-001 | 20 | NR | NR | NR | 2635.63 | NR |

Longer half-life 16.42 vs 8.84 h, slightly lower Cmax 278.23 vs 353 ng/mL, slightly lower AUC last 1710 vs 1701.33 and slightly higher AUC inf 2355.81 vs 2034 (ng*h/mL), lower AUC at Day 15 2635.63 vs 3498 (ng*h/mL).

### 3.4. PCLX-001 Toxicities

The patient diary revealed perfect compliance with the medication delivery schedule. The patient experienced no protocol defined dose limiting toxicities. Her hematopoietic profile and biochemistry remained normal throughout the trial.

### 3.5. Efficacy

The subject had baseline cross sectional and PET imaging. Due to increasing patient symptoms of back pain, repeat computed tomography imaging was performed on day 28 and revealed progression of the volume of lymphoma as defined by the Lugano classification criteria for the evaluation of non-Hodgkin lymphoma. In consequence, the patient discontinued PCLX-001 at that time.

### 4. Results-Patient 2

### 4.1. Patient Demographics

This patient (Patient 2) was a 56 year-old female with transformed Stage IV DLBCL diagnosed in April 2018, initially treated with 6 cycles of R-CEOP immunochemotherapy which was completed in September 2018 with complete response. The lymphoma relapsed in May 2019 and she received R-DICEP salvage immunochemotherapy followed by BEAM high dose conditioning chemotherapy with Autologous Stem Cell Transplant. Progressive lymphoma was managed in mid-2020 with an anti-CD19 CAR-T protocol. Progressive lymphoma was diagnosed in May 2021. The patient received intermittent radiotherapy and corticosteroids. She consented to be the second patient to receive PCLX-001 as part of the PCLX-001 clinical trial in October 2021.

### 4.2. PCLX-001 Administration

PCLX-001 was administered orally each morning as two tablets, each containing PCLX-001 10 mg. PCLX-001 was initiated on Nov. 9, 2021 and discontinued on day Jan. 3, 2022 due to mixed disease response to therapy as demonstrated by an interval marked tumor response in hepatic metastases but progressive disease in lymph nodes; discontinuation of study drug was recommended in the setting of mixed disease response by the treating clinician to permit other therapies.

### 4.3. Pharmacokinetics

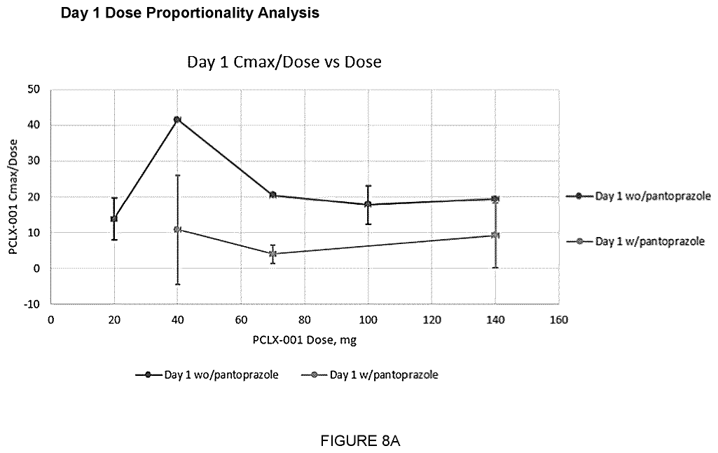

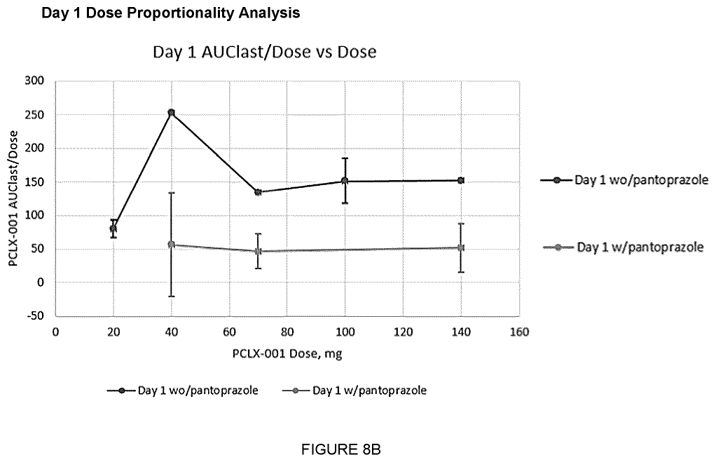

The pharmacokinetic analysis was conducted as described above. FIG. 4 shows the plasma concentration of PCLX-001 after the initial oral dose. Table 1A and Table 1B shows the pharmacokinetic parameters determined by this analysis, where she is described as patient 115-002-002. After her first PCLX-001 dose, peak plasma concentration was achieved at 4 hours, and the peak plasma concentration was 127 ng/ml. With day 15 dosing, peak plasma concentration was achieved after 2 hours, peak plasma concentration was 215 ng/ml, and terminal half-life was estimated at 10.3 h. Nonparametric superposition simulations (FIG. 8) show rapid achievement of steady state kinetics.

In Patient 2 we observed efficacy of the dosing regimen and therapeutic utility. Daily oral administration of 20 mg PCLX-001 tablets achieved a lower Cmax and longer Tmax and a lower extent of exposure were apparent compared to the patient 1. The pharmacokinetic parameters were all supportive of a single daily oral dosing schedule for PCLX-001 but could also be amenable to more frequent daily dosing, or a modified release formulation.

In a finding unexpected and unanticipated by allometric scaling of PK results in mice, rats, and dogs, PCLX-001 dosing consistently achieved trough concentrations (those measured immediately prior to dosing on day 2, 15, and 22) higher than anticipated in each of patient 1, patient 2, and patient 3. Peak plasma concentrations exceeded those needed to suppress cultured human cancer cell growth. Trough plasma concentrations approached those needed to suppress cultured human cancer cell growth.

### 4.3.1 Computed Tomography

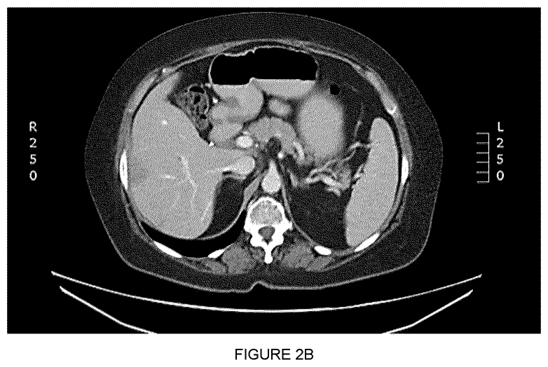

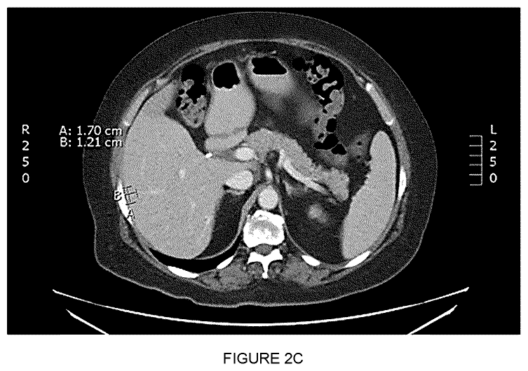

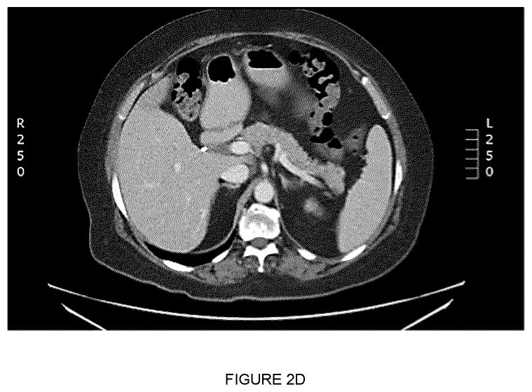

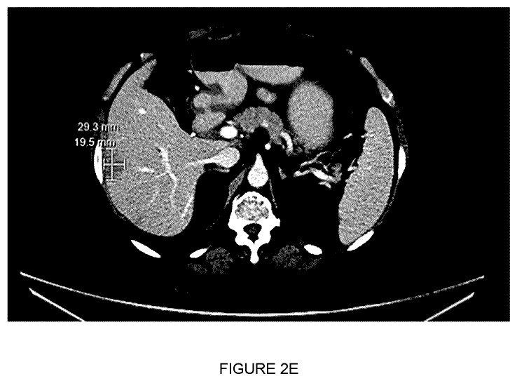

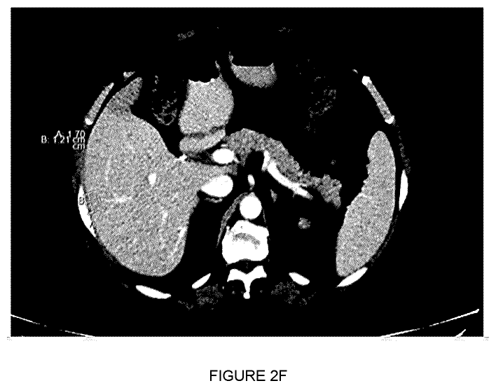

Computed tomography was performed on patient 2 on two occasions, immediately prior to treatment (pre-treatment scan obtained Oct. 29, 2021) and after two 28-day cycles of oral PCLX-001 at a daily dose of 20 mg by mouth (post-treatment scan obtained Dec. 30, 2021). Both scans were done on the same scanner using the same techniques including the use of intravenous contrast dye. The three pairs of pre and post treatment scans are of the same cross section of the patient, in order to identify a clinical trial target lesion of a liver metastasis in the subcapsular region of segment five of the liver. These scans demonstrate a baseline discrete 2.9×2 cm liver metastasis at baseline. These scans demonstrate post-treatment a vague questionable residual hypoattenuation measuring 1.7×1.2 cm. These radiologic changes indicated marked tumor regression. FIG. 2A-4F.

### 4.5. PCLX-001 Toxicities

The patient diary revealed perfect compliance with the medication delivery schedule. The patient experienced no protocol defined dose limiting toxicities. Her hematopoietic profile and biochemistry remained normal throughout the trial.

### 4.6 Efficacy

As shown herein, an unexpectedly high drug exposure was achieved with PCLX-001, which is evident by higher than anticipated trough concentrations (i.e., the drug levels measure right before taking the next pill, FIGS. 5-7), the longer than anticipated half-life compared to animal studies, and evidence that patient number 2 had regression of a malignant lesion in the liver (FIG. 2A-F).

### 4.7 Summary of Patient 3 Demographics and Results

This patient was a 55 year old female with a heavily pretreated leiomyosarcoma with systemic metastases. She received daily PCLX-001 from 24 Nov. 2021 through 19 Jan. 2022. No drug related toxicities were identified. Her medication record showed perfect compliance with her medications. Response assessment scans after two 28 day cycles of PCLX-001 showed progressive disease, and study medication was discontinued. Her pharmacokinetic results are summarized in Table 1A and Table 1B (where she is represented as Patient 115-002-004).

### 4.8 Serum PCLX-001 Levels Expected for Efficacy

Preclinical data on ex-vivo sensitivity of various cultured human cancer cells has been performed in three panels and previously reported (3). Table 2 summarizes the mean concentrations that suppressed cancer cell growth among sensitive cancer cell lines (defined as EC50<500 nM or IC50<500 nM). As can be seen, mean effective concentrations of PCLX-001 were achieved at concentrations as low as low as 80.9 nm. These levels are exceeded by peak plasma concentrations in each of patients 1, 2 and 3. The trough concentrations observed on day 2, day 15, and day 22 are also close to those required to suppress cancer cell growth (FIGS. 5, 6, 7). The conversion factor from ng/ml to nM for PCLX-001 is a multiplier of approximately 1.86, where, for example, 100 ng/ml converts to a concentration of 186 nM.

TABLE 2
In vitro inhibitory concentrations of PCLX-001
in three panels of cultured human cancer cells.

| | (EC50 nM) | (IC50 nM) | (IC50 nM) |
|---|---|---|---|
| | mean +/− SD | mean +/− SD | mean +/− SD |
| Bladder | 154.9 ± 49.7 | 171.9 ± 79.8 | |
| Brain | | 153.3 ± 63.5 | |
| Breast | 101.4 ± 96.4 | 161.0 ± 47.9 | |
| Colon | | 157.5 ± 27.4 | 210.5 ± 85.4 |
| Leukemia | 97.9 ± 81.3 | 189.4 ± 87.8 | |
| Lung | 150.6 ± 88.2 | 184.5 ± 82.8 | 153.6 ± 111.4 |
| Lymphoma | 80.9 ± 63.5 | 138.3 ± 55.8 | 230.1 ± 107.4 |
| Melanoma | 133.6 ± 53.9 | 207.6 ± 99.5 | 156.9 ± 191.6 |
| Myeloma | 143.9 ± 44.7 | | |
| Neuroblastoma | | 230.0 ± 182.7 | |
| Ovary | | 76.7 ± 7.6 | |
| Pancreas | 109.1 ± 90.0 | 77.7 ± 13.7 | |
| Prostate | | 85.4 ± 59.2 | |
| Sarcoma | | 197.5 ± 133.7 | |
| Stomach | | 120.0 ± 48.2 | |

### 4.9 Trough Levels of PCLX-001 are Higher than Expected

The summary of selected previous pharmacokinetic analyses performed on non-human species are shown in Table 3. These studies showed that PCLX-001 was rapidly eliminated from plasma in non-human species. Consequently, the consistent finding of substantial PCLX-001 plasma levels prior to dosing on day 2, day 8, day 15, and day 22, was an unexpected finding in all patients (FIGS. 5-7). Possible explanations for this finding include hysteresis loops (7), and flip-flop pharmacokinetics (7).

### 4.10 Allometric Scaling from Animal Studies Suggest PCLX-001 Oral Doses Needed to Achieve Efficacy Must be Substantially Higher than 20 mg Daily

Allometric scaling is a means by which PK results from animal studies can be used to predict human drug exposure for a range of drug doses. In this case, allometric scaling was performed using representative values from the PK studies with daily oral PCLX-001 administration for 14 or 28 days in non-human species. These inputs were analyzed with www.truedose.app to predict the Maximally Tolerated Dose we would see in this phase one clinical trial (FIG. 2). Anticancer efficacy data from mice bearing human tumors suggested that the effective dose of PCLX-001 was greater than 50% of the PCLX-001 dose that produced mouse toxicity in any given experiment.

TABLE 3
Allometric scaling from mouse, rat, dog maximally tolerated doses
to predict human PCLX-001 daily oral maximally tolerated doses.

| | | | Maximally | Allometric |
|---|---|---|---|---|
| | | Terminal | Tolerated Dose | prediction of |
| | Cmax | elimination | daily oral | human MTD |
| Species | (ng/ml) | half-life (h) | administration | (mg/70 kg) |
| mouse | 46000 | 1.6 | 35 mg/kg | 180 mg |
| rat | 3110 | 1.0 | 125 mg/kg | 1300 mg |
| dog | 650 | 3.8 | 4 mg/kg | 137 mg |
| human | 479 | 16.0 | TBD | Not applicable |
| (patient 1) | | | (20 mg/75 kg = | |
| | | | 0.27 mg/kg) | |

In aggregate, the animal PK data, animal efficacy data, and animal allometric scaling suggest that PCLX-001 20 mg administered orally daily would not be expected to achieve anticancer effects. Consequently, the human clinical trial results are unanticipated and novel.

FIG. 1 depicts the chemical structure of PCLX-001 (2,6-dichloro-N-(3-isobutyl-1,5-dimethyl-1H-pyrazol-4-yl)-4-(2-(piperazin-1-yl) pyridin-4-yl)benzenesulfonamide) C₂₄H₃₀Cl₂N₆O₂S.

FIG. 2 depicts computed tomography images of Patient 2 (A) Oct. 29, 2021—TL #2 Segment 5 of Liver, (B) Oct. 29, 2021—TL #2 Segment 5 of Liver (w/o measurements) Image 27/4, (C) Dec. 30, 2021—TL #2 Segment 5 of Liver Image 24/4 with measurements, (D) Dec. 30, 2021—TL #2 Segment 5 of Liver (w/o measurements Image 24/4, (E) Oct. 29, 2021—TL #2 Segment 5 of Liver Image 27/4 (Liver Views), (F) Dec. 30, 2021—TL #2 Segment 5 of Liver Image 24/4 (Liver Views).

FIG. 3 depicts non-compartmental analysis for PCLX-001 in Patient 1 (Patient 115-002-001) (A) day 1 (B) day 15.

FIG. 4(A) depicts non-compartmental analysis for PCLX-001 in Patient 2 (Patient 115-002-002) day 1, (B) day 15, (C) depicts non-compartmental analysis for PCLX-001 in Patient 3 (Patient 115-002-004) day 1, (D) day 15.

FIG. 5 depicts nonparametric superposition simulations to steady state in Patient 1 (Patient 115-002-001).

FIG. 6 depicts nonparametric superposition simulations to steady state in Patient 2 (Patient 115-002-002).

FIG. 7 depicts nonparametric superposition simulations to steady state in Patient 3 (Patient 115-002-004).

FIG. 8A and FIG. 8B depict day 1 dose proportionality analysis. FIG. 8A depicts D1 Cmax/Dose vs Dose. FIG. 8B depicts Day 1 AUClast/Dose vs Dose.

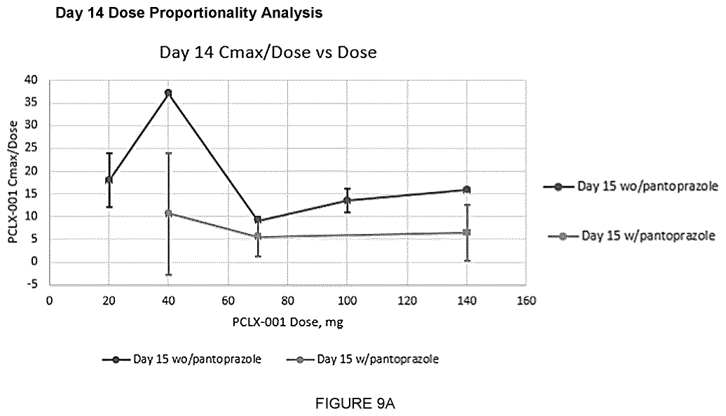

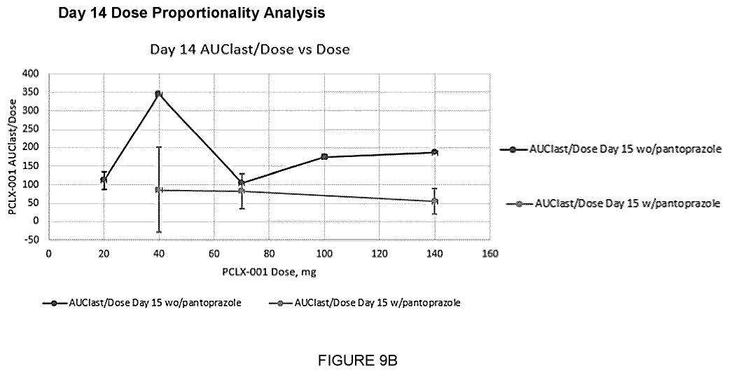

FIG. 9A and FIG. 9B depict Day 14 Dose Proportionality Analysis. FIG. 9A depicts Day 14 Cmax/Dose vs Dose. FIG. 9B depicts Day 14 AUClast/Dose vs Dose.

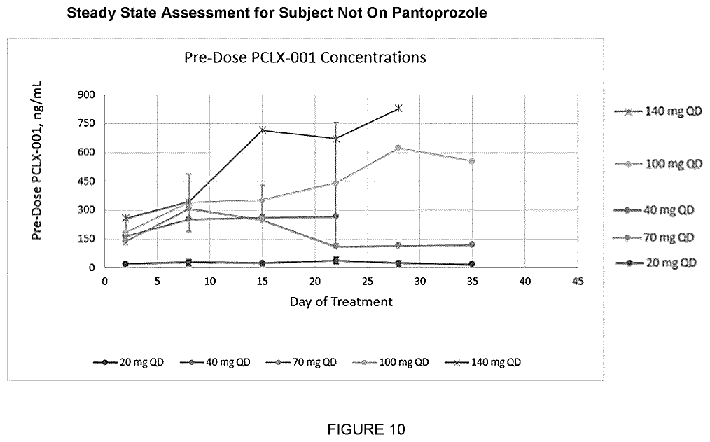

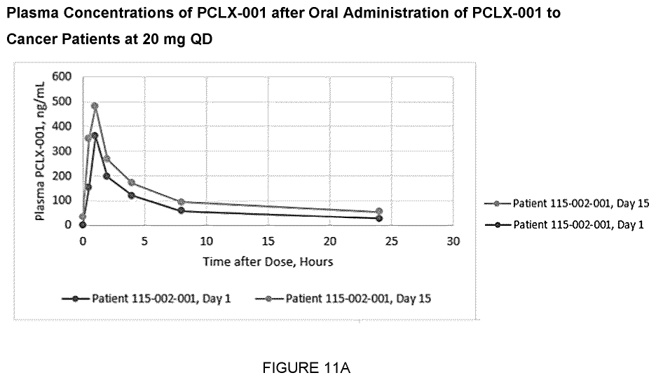

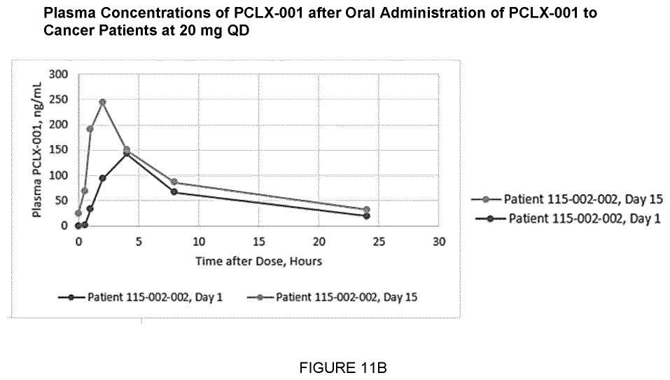

FIG. 10 depicts Steady State Assessment for Subject Not on Pantoprazole Pre-Dose PCLX-001 Concentrations.

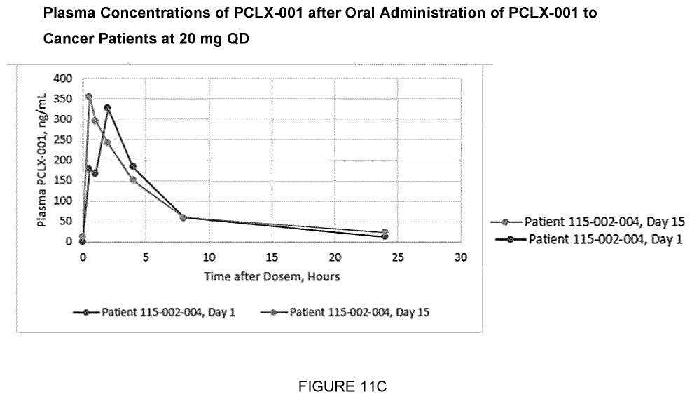

FIG. 11A, FIG. 11B, and FIG. 11C, depict Plasma Concentrations of PCLX-001 after Oral Administration of PCLX-001 to Cancer Patients at 20 mg QD.

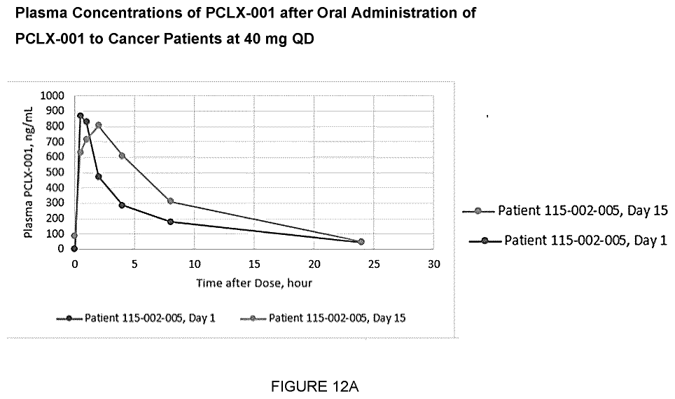

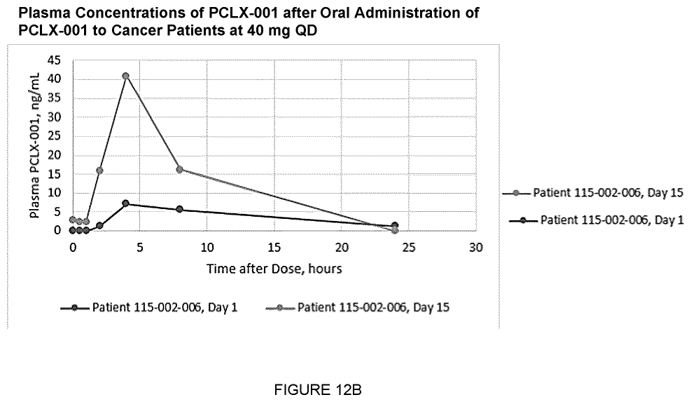

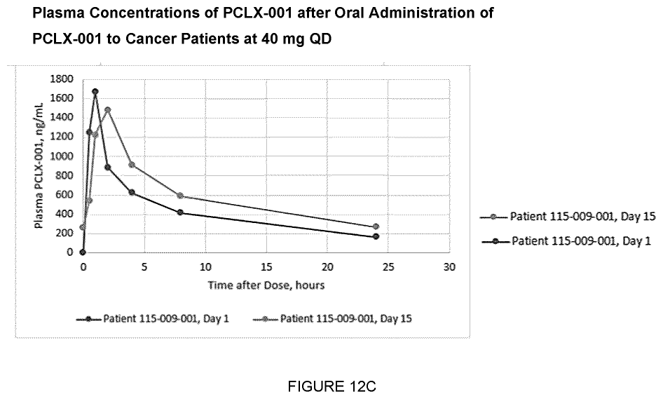

FIG. 12A, FIG. 12B, and FIG. 12C, depict Plasma Concentrations of PCLX-001 after Oral Administration of PCLX-001 to Cancer Patients at 40 mg QD.

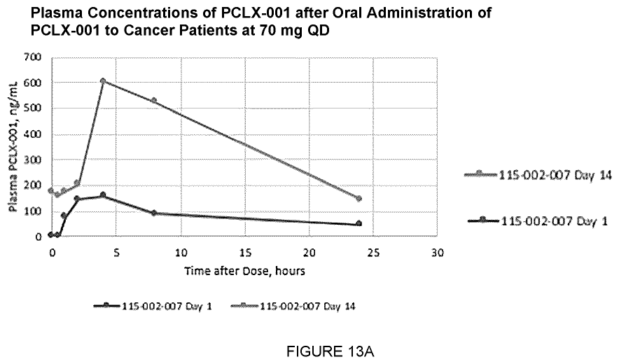

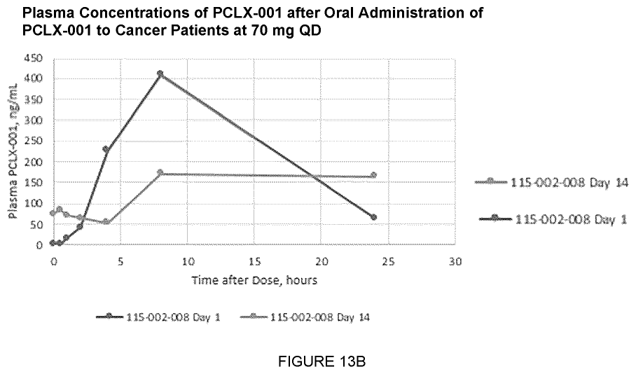

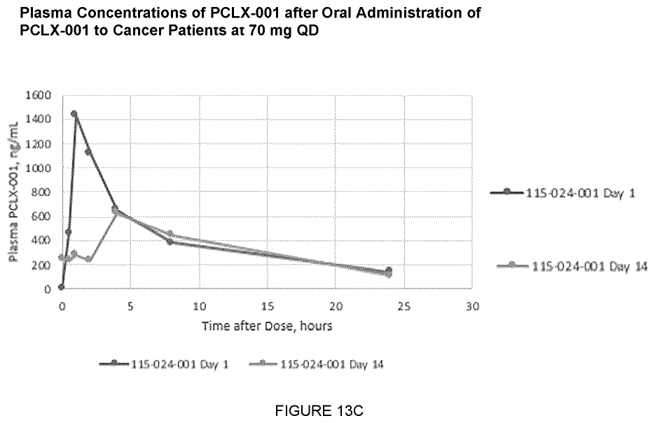

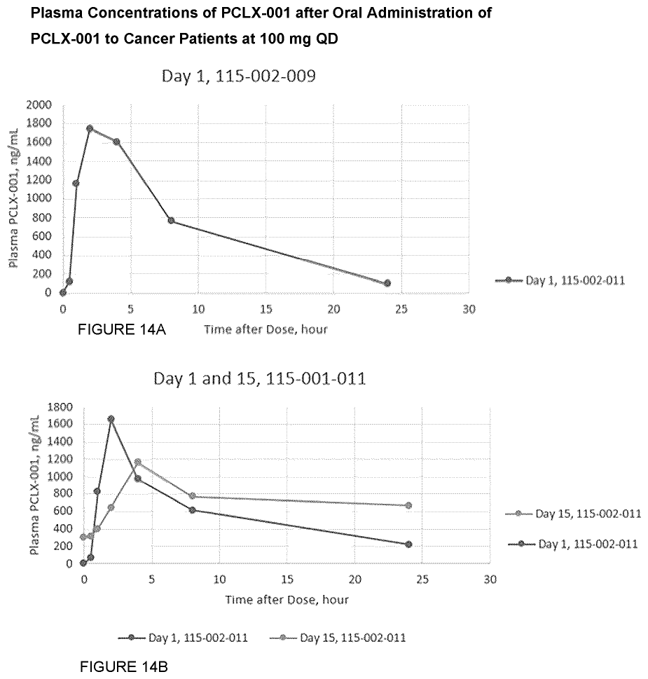

FIG. 13A, FIG. 13B, FIG. 13C, depicts Plasma Concentrations of PCLX-001 after Oral Administration of PCLX-001 to Cancer Patients at 70 mg QD.

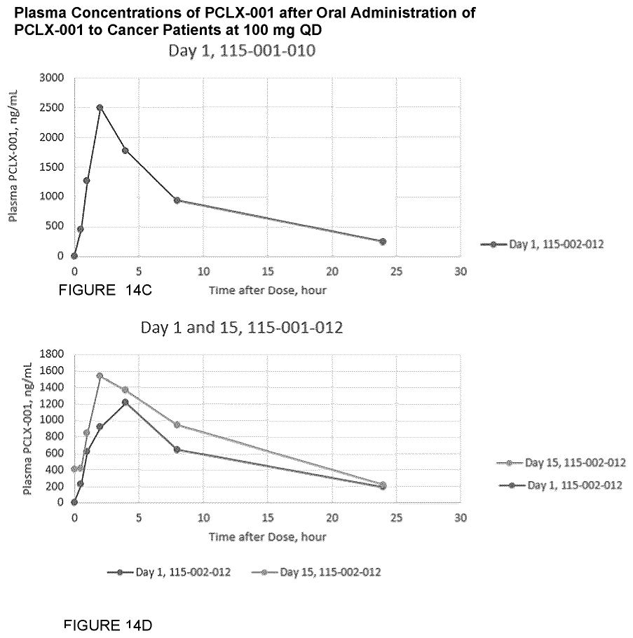

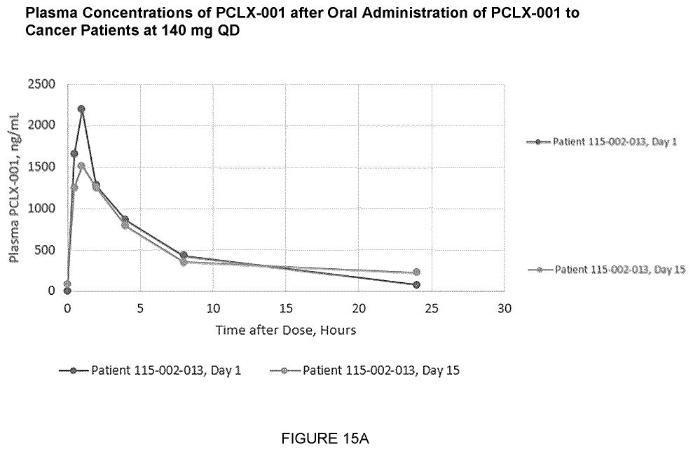

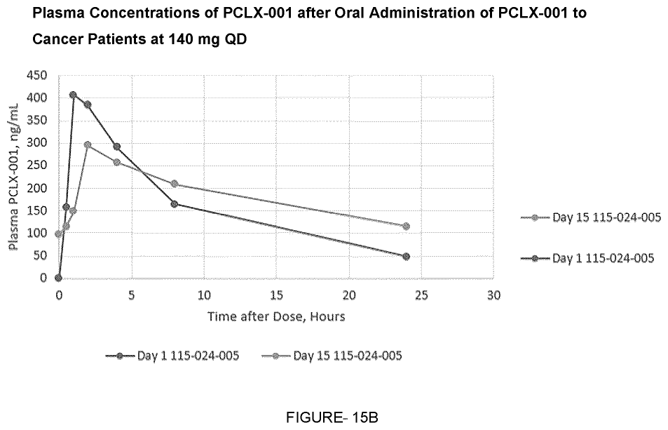

FIG. 14A, FIG. 14B, FIG. 14C, FIG. 14D depicts Plasma Concentrations of PCLX-001 after Oral Administration of PCLX-001 to Cancer Patients at 100 mg QD.

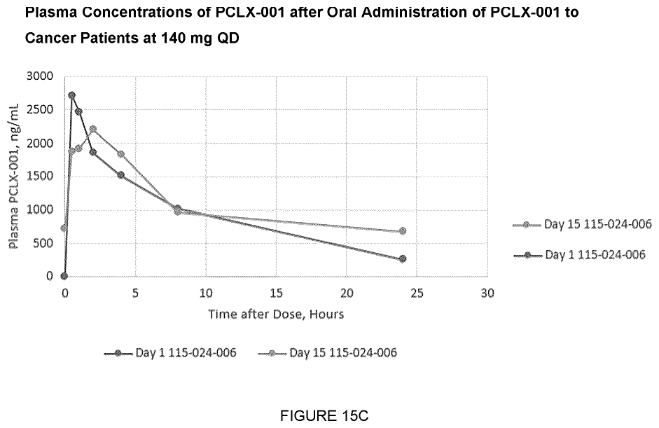

FIG. 15A, FIG. 15B, FIG. 15C, depicts Plasma Concentrations of PCLX-001 after Oral Administration of PCLX-001 to Cancer Patients at 140 mg QD.

In the non-compartmental analysis, Phoenix WinNonlin 8.3 trapezoidal rule was used, with linear up log down method. This allowed WinNonlin to pick optimal terminal phase (WinNonlin will accept Rsq adjusted to >0.7). Additional analysis to assess adequate fie included visual inspection of data, and AUCₑₓₜᵣₐₚ should be <15%.

In the nonparametric superposition, Phoenix WinNonlin Nonparametric Superposition used to predict steady state data based upon Day 1 data for each patient. The 8- and 24-hour time points were utilized for estimation of terminal phase for the purposes of this exercise. Simulated 20 mg QD. Simulations were conducted out to steady state.

### 5. Discussion

This is the first report of human exposure to a potential new class of anti-cancer drug, inhibitors of N-myristoyltransferase. In this prospective, phase I dose escalation study, the first patient received daily 20 mg oral PCLX-001 for a complete 28 day cycle and experienced no dose limiting toxicities. This patient had relapsed and pretreated aggressive non-Hodgkin lymphoma, and although she experienced disease progression despite PCLX-001, there was no evidence of toxicity to hematopoietic function, liver function, or kidney function.

In Patient 1 the pharmacokinetic analysis was conducted as described above. FIG. 3 shows the plasma concentration of PCLX-001 after the initial oral dose and after day 15 dosing. Table 1A and Table 1B shows the pharmacokinetic parameters determined by this analysis. Day 1 results show a rapid absorption rate, with peak plasma concentration achieved at 1 hours after oral administration, a peak plasma concentration of 353 ng/ml, and an apparent terminal half-life of 8.5 hours. Day 15 results show rapid absorption with a peak plasma concentration of 479 ng/mL achieved at 1 hour after administration. Nonparametric superposition simulations (FIG. 5) show rapid achievement of steady state kinetics. These parameters are all supportive of a single daily oral dosing schedule for PCLX-001.

Furthermore, the plasma trough levels, as measured immediately before daily dosing, achieve drug concentrations (FIGS. 5-7) that approximate the IC50 required to inhibit some PCLX-001-sensitive cultured cancer cell lines.

Patient 1 had prior exposure to the Bruton tyrosine kinase (BTK) inhibitor ibrutinib. Some of myristoylation substrates for NMTs, including Lyn and HGAL, are upstream of BTK in the B-cell receptor signaling cascade [3]. Whether prior BTK inhibitors relate to PCLX-001 efficacy will be assessed as additional patients are accrued to this ongoing study.

For patient 2, the pharmacokinetic analysis was conducted as described above. FIG. 5 shows the plasma concentration of PCLX-001 after the initial oral dose. Table 1A Table 1B shows the pharmacokinetic parameters determined by this analysis, where she is described as patient 115-002-002. After her PCLX-001 dose, peak plasma concentration was achieved at 4 hours, and the peak plasma concentration was 127 ng/ml. With day 15 dosing, peak plasma concentration was achieved after 2 hours, peak plasma concentration was 215 ng/ml, and terminal half-life was estimated at 10.3 h. Nonparametric superposition simulations show rapid achievement of steady state kinetics. We noted lower extent of PCLX-001 exposure, compared to Patient 1. Nonetheless, the drug exposure and trough levels of PCLX-001 (FIG. 6) were unexpectedly high when compared to the expectations from pre-clinical studies in animals. After continuous daily oral administration of PCLX-001 for approximately two months, Patient number 2 had regression of a malignant lesion in the liver (FIG. 2).

### 6. Conclusions

This is the first report of any therapeutic human use of N-myristoyltransferase inhibitors. PCLX-001, a potent, small molecule inhibitor of human N-myristoyltransferase proteins, had favorable pharmacokinetic parameters suitable for once daily oral dosing, or more frequent oral dosing, or a modified release formulation. Unexpectedly, low doses of this drug produced higher-than-expected drug exposures and radiologic regression of a malignant hepatic metastasis in a woman with heavily pretreated DLBCL.

### REFERENCES

1. Castrec, B.; Dian, C.; Ciccone, S.; Ebert, C. L.; Bienvenut, W. V.; Le Caer, J. P.; Steyaert, J. M.; Giglione, C.; Meinnel, T. Structural and genomic decoding of human and plant myristoylomes reveals a definitive recognition pattern. Nat Chem Biol 2018, 14, 671-679, doi: 10.1038/s41589-018-0077-5.
2. Selvakumar, P.; Lakshmikuttyamma, A.; Shrivastav, A.; Das, S. B.; Dimmock, J. R.; Sharma, R. K. Potential role of N-myristoyltransferase in cancer. Prog Lipid Res 2007, 46, 1-36, doi: 10.1016/j.plipres.2006.05.002.
3. Beauchamp, E.; Yap, M. C.; Iyer, A.; Perinpanayagam, M. A.; Gamma, J. M.; Vincent, K. M.; Lakshmanan, M.; Raju, A.; Ter-gaonkar, V.; Tan, S. Y.; et al. Targeting N-myristoylation for therapy of B-cell lymphomas. Nat Commun 2020, 11, 5348, doi: 10.1038/s41467-020-18998-1.
4. Mackey, J. R.; Lai, J.; Chauhan, U.; Beauchamp, E.; Dong, W. F.; Glubrecht, D.; Sim, Y. W.; Ghosh, S.; Bigras, G.; Lai, R.; et al. N-myristoyltransferase proteins in breast cancer: prognostic relevance and validation as a new drug target. Breast Cancer Res Treat 2021, 186, 79-87, doi: 10.1007/s10549-020-06037-y.
5. Weickert, M.; Dillberger, J.; Mackey, J. R.; Wyatt, P.; Gray, D.; Read, K.; Li, C.; Parenteau, A.; Berthiaume, L. G. Initial Characterization and Toxicology of an Nmt Inhibitor in Development for Hematologic Malignancies. Blood 2019, 134, 3362-3362, doi: 10.1182/blood-2019-124934.
6. Sangha, R.; Mackey, J. R.; Sehn, L. H.; Kuruvilla, J.; Weickert, M. J.; Berthiaume, L. G. An Open-Label, First-in-Human, Phase I Trial of Daily Pclx-001. Blood 2021, 138, 1364-1364, doi: 10.1182/blood-2021-153613.
7. Louizos, C., Yanez, J. A., Forrest, M. L., Davies, N. M. J Pharm Pharm. Sci. 2014; 17 (1): 34-91
8. Yanez, J. A., Remsberg, C. M., Sayre, C. L., Forrest, M. L., Davies, N. M. Ther Deliv. 2011 May; 2 (5): 643-672. Doi: 10.4155/tde. 11.19.

### Example 2

In this example it is shown that PCLX-001 was readily absorb with median (range) Tmax values of 2.00 (0.50, 8.00). Tmax values were similar on Days 1 and Day 15 of dosing.

The t1/2 of PCLX-001 ranged from 5.02 to 17.4 hours across the dose groups. The t1/2 of PCLX-001 was similar between patients on pantoprazole and those patients who were not on pantoprazole. The t1/2 tended to be higher in the highest dose group (140 mg).

The systemic exposure to PCLX-001 was dose proportional over the dose range of 20 to 140 mg.

The systemic exposure to PCLX-001 was lower in patients taking pantoprazole.

TABLE 4
Mean (CV) Pharmacokinetic Parameters for PCLX-001 Following Daily
Oral Administration of PCLX-001 to Patients not on Pantoprazole

| Dose, | | | t½, | Tₘₐₓ | Cₘₐₓ, | AUClast, | | AUCinf, | |
|---|---|---|---|---|---|---|---|---|---|
| mg | N | Day | hour | hour | ng/mL | ng*h/mL | | ng*h/mL | |
| 20 | 3 | 1 | 6.93 (29) | 2.00 (1.00, 4.00) | 276 (43) | 1602 | (16) | 1956 | (6) |
| 20 | 3 | 15 | 8.93 (25) | 1.00 (0.50, 2.00) | 359 (33) | 2221 | (21) | 2717 | (29) |
| 40 | 1 | 1 | 10.8 | 1.00 | 1670 | 10099 | | 12633 | |
| 40 | 1 | 15 | 11.9 | 2.00 | 1480 | 13748 | | 18306 | |
| 70 | 1 | 1 | 9.44 | 1.00 | 1430 | 9395 | | 11273 | |
| 70 | 1 | 15 | NC | 4.00 | 629 | 7301 | | NC | |
| 100 | 4 | 1 | 7.26 | 2.00 (2.00, 4.00) | 1773 (30) | 15140 | (22) | 18016 | (21) |
| 100 | 2 | 15 | 7.56* | 3.00 (2.00, 4.00) | 1345 (20) | 17444 | (3) | 19387* | |
| 140 | 1 | 1 | 7.91 | 0.50 | 2710 | 21234 | | 24165 | |
| 140 | 1 | 15 | 13.8 | 1.50 (1.00, 2.00) | 903 (95) | 7681 | (62) | 10738 | (47) |

NC = Not calculated as elimination rate could not be adequately estimated.
Tmax is expressed as median (min, max).
*Represents value from N = 1 patient.

TABLE 5
Mean (CV) Pharmacokinetic Parameters for PCLX-001 Following Daily
Oral Administration of PCLX-001 to Patients on Pantoprazole

| Dose, | | | t½, | Tₘₐₓ, | Cₘₐₓ, | AUClast, | AUCinf, |
|---|---|---|---|---|---|---|---|
| mg | N | Day | hour | hour | ng/mL | ng*h/mL | ng*h/mL |
| 40 | 1 | 1 | 7.5 | 0.50 | 868 | 4431 | 4898 |
| 40 | 1 | 15 | 5.54 | 2.00 | 802 | 6679 | 7055 |
| 70 | 2 | 1 | NC | 6.00 (4.00, 8.00) | 283 (63) | 3255 (56) | NC |
| 70 | 2 | 15 | NC | 6.00 (4.00, 8.00) | 386 (79) | 5757 (58) | NC |
| 140 | 2 | 1 | 6.71 | 1.00 (1.00, 1.00) | 1298 (97) | 7257 (71) | 7832 (66) |
| 140 | 2 | 15 | 13.76 | 1.50 (1.00, 2.00) | 903 (95) | 7681 (62) | 10738 (47) |

NC = Not calculated as elimination rate could not be adequately estimated.
Tₘₐₓ is expressed as median (min, max).

In one example, the T1/2 is about 5 hours to about 18 hours. In another example, the T1/2 is about 5.5 hours to about 14 hours.

In one example, where the oral does comprises 20 mg PCLX-001 per day, the T1/2 is about 6 hours to about 9 hours. In another example, the T1/2 is about 6.9 hours to about to about 8.9 hours.

In one example, therein the oral dose comprises 40 mg PCLX-001 per day, the T1/2 is about 5 hours to about 11 hours. In another example, the T1/2 is about 5.5 hours to about 10.8 hours.

In one example, therein the oral dose comprises 70 mg PCLX-001 per day, the T1/2 is about 9 hours. In another example, the T1/2 is about 9.4 hours.

In one example, therein the oral dose comprises 100 mg PCLX-001 per day, the T1/2 is about 7 hours to about 8 hours. In another example, the T1/2 is about 7.2 hours to about 7.5 hours.

In one example, therein the oral dose comprises 140 mg PCLX-001 per day, the T1/2 is about 6 hours to about 14 hours. In another example, the T1/2 is about 6.7 hours to about 13.8 hours.

FIGS. 8A and 8B depict day 1 dose proportionality analysis. FIG. 8A depicts D1 Cmax/Dose vs Dose. FIG. 8B depicts Day 1 AUClast/Dose vs Dose. Patients on pantoprazole had lower PCLX-001 Cmax and AUClast values than patients not taking pantoprazole. Consistent AUCtau/dose or Cmax/dose values across the dose range of 20 mg to 140 mg QD suggests dose proportionality.

FIGS. 9A and 9B depict Day 14 Dose Proportionality Analysis. FIG. 9A depicts Day 14 Cmax/Dose vs Dose. FIG. 9B depicts Day 14 AUClast/Dose vs Dose. Patients on pantoprazole had lower PCLX-001 Cmax and AUClast values than patients not taking pantoprazole. Consistent AUCtau/dose or Cmax/dose values across the dose range of 20 mg to 140 mg QD suggests dose proportionality.

FIG. 10 depicts Steady State Assessment for Subject Not On Pantoprazole-Pre-Dose PCLX-001 Concentrations. Data are limited to assess time to achieve steady state due to low subject number for 20 mg (N=3), 40 mg (N=1), 70 mg (N=1), 100 mg (N=2) and 140 mg (N=1).

### Supporting Individual Data Slides

### Noncompartmental Analysis

Phoenix WinNonlin 8.3 trapezoidal rule. Used linear up log down method. Allowed WinNonlin to pick optimal terminal phase (WinNonlin will accept Rsq adjusted >0.7).

Other analysis to assess adequate fit. Visual inspection of data. AUCₑₓₜᵣₐₚ should be <15%.

Plots. In all plots Day 22 pre-dose value was used for the Day 15, 24 hour value as value was not collected as 24 hour sample for Day 15 was not collected

FIGS. 11A, 11B, and 11C, depict Plasma Concentrations of PCLX-001 after Oral Administration of PCLX-001 to Cancer Patients at 20 mg QD.

TABLE 6
Plasma PK Parameters for PCLX-001 20 mg QD Oral.

| | | | | | | AUCₗₐₛₜ or | | | |
|---|---|---|---|---|---|---|---|---|---|
| | | Dose | Half-life | Tₘₐₓ | Cₘₐₓ | AUCₜₐᵤ, | AUC_{inf} | AUCₑₓₜᵣₐₚ | |
| Day | Patient | (mg) | (h) | (h) | (ng/mL) | (ng*h/mL) | (ng*h/mL) | (%) | Pantoprazole |
| 1 | 115-002-001 | 20 | 8.34 | 1 | 360 | 1721 | 2031 | 15.3 | No |
| 1 | 115-002-002 | 20 | * | 4 | 142 | 1300 | * | * | No |
| 1 | 115-002-004 | 20 | 5.51 | 2 | 326 | 1785 | 1880 | 5.1 | No |
| 15 | 115-002-001 | 20 | 10.9 | 1 | 479 | 2748 | 3602 | 23.7 | No |
| 15 | 115-002-002 | 20 | 9.42 | 2 | 243 | 2014 | 2441 | 17.5 | No |
| 15 | 115-002-004 | 20 | 6.47 | 0.5 | 356 | 1900 | 2111 | 10.0 | No |

*The terminal half-life and AUCinf could not be adequately characterized.
Note:
The AUCextrap highlighted in red and underlined if >15% of AUC is extrapolated.

FIGS. 12A, 12B, and 12C, depict Plasma Concentrations of PCLX-001 after Oral Administration of PCLX-001 to Cancer Patients at 40 mg QD. Note: Day 22 pre-dose value was plotted for visualization as Day 15, 24 hour sample was not collected.

TABLE 7
depicts Plasma PK Parameters for PVCLX-001 40 mg QD Oral.

| | | | | | | AUCₗₐₛₜ or | | | |
|---|---|---|---|---|---|---|---|---|---|
| | | Dose | Half-life | Tₘₐₓ | Cₘₐₓ | AUCₜₐᵤ, | AUC_{inf} | AUCₑₓₜᵣₐₚ | |
| Day | Patient | (mg) | (h) | (h) | (ng/mL) | (ng*h/mL) | (ng*h/mL) | (%) | Pantoprazole |
| 1 | 115-002-005 | 40 | 7.50 | 0.5 | 868 | 4432 | 4899 | 9.5 | Yes |
| 1 | 115-002-006 | 40 | * | 4 | 7.13 | 79.6 | * | | Yes |
| 1 | 115-009-001 | 40 | 10.8 | 1 | 1670 | 10099 | 12633 | 20.1 | No |
| 15 | 115-002-005 | 40 | 5.54 | 2 | 802 | 6679 | 7055 | 5.3 | Yes |
| 15 | 115-002-006 | 40 | * | 4 | 40.8 | 175 | * | | Yes |
| 15 | 115-009-001 | 40 | 11.9 | 2 | 1480 | 13749 | 18306 | 24.9 | No |

*The terminal half-life and AUCinf could not be adequately characterized.
Note:
The AUCextrap highlighted in red and underlined if >15% of AUC is extrapolated

FIGS. 13A, 13B, 13C, depicts Plasma Concentrations of PCLX-001 after Oral Administration of PCLX-001 to Cancer Patients at 70 mg QD.

TABLE 8
depicts Plasma PK Parameters for PVCLX-001 70 mg QD Oral.

| | | | | | | AUCₗₐₛₜ or | | | |
|---|---|---|---|---|---|---|---|---|---|
| | | Dose | Half-life | Tₘₐₓ | Cₘₐₓ | AUCₜₐᵤ, | AUC_{inf} | AUCₑₓₜᵣₐₚ | |
| Day | Patient | (mg) | (h) | (h) | (ng/ml) | (ng*h/mL) | (ng*h/mL) | (%) | Pantoprazole |
| 1 | 115-002-007 | 70 | * | 4 | 158 | 1963 | * | * | Yes |
| 1 | 115-002-008 | 70 | * | 8 | 408 | 4546 | * | * | Yes |
| 1 | 115-024-001 | 70 | 9.44 | 1 | 1430 | 9395 | * | 16.8 | No |
| 15 | 115-002-007 | 70 | * | 4 | 602 | 8134 | * | * | Yes |
| 15 | 115-002-008 | 70 | * | 8 | 170 | 3379 | * | * | Yes |
| 15 | 115-024-001 | 70 | * | 4 | 629 | 7301 | * | * | No |

*The terminal half-life and AUCinf could not be adequately characterized.
Note:
The AUCextrap highlighted in red and underlined if >15% of AUC is extrapolated.

FIGS. 14A, 14B, 14C, 14D depicts Plasma Concentrations of PCLX-001 after Oral Administration of PCLX-001 to Cancer Patients at 100 mg QD.

TABLE 9
Plasma PK Parameters for PVCLX-001 100 mg QD Oral.

| | | | | | | AUCₗₐₛₜ or | | | |
|---|---|---|---|---|---|---|---|---|---|
| | | Dose | Half-life | Tₘₐₓ | Cₘₐₓ | AUCₜₐᵤ, | AUC_{inf} | AUCₑₓₜᵣₐₚ | |
| Day | Patient | (mg) | (h) | (h) | (ng/mL) | (ng*h/mL) | (ng*h/mL) | (%) | Pantoprazole |
| 1 | 115-002-009 | 100 | 5.02 | 2 | 1740 | 14759 | 15444 | 4.4 | No |
| 1 | 115-002-010 | 100 | 7.17 | 2 | 2490 | 19959 | 22389 | 10.9 | No |
| 1 | 115-002-011 | 100 | 9.60 | 2 | 1650 | 13224 | 16216 | 18.4 | No |
| 1 | 115-002-012 | 100 | * | 4 | 1210 | 12616 | * | * | No |
| 15 | 115-002-011 | 100 | * | 4 | 1160 | 17867 | * | * | No |
| 15 | 115-002-012 | 100 | 7.56 | 2 | 1530 | 17020 | 19387 | 12.2 | No |

*The terminal half-life and AUCinf could not be adequately characterized.
Note:
The AUCextrap highlighted in red and underlined if >15% of AUC is extrapolated.

FIGS. 15A, 15B, 15C, depicts Plasma Concentrations of PCLX-001 after Oral Administration of PCLX-001 to Cancer Patients at 140 mg QD.

TABLE 10
Plasma PK Parameters for PCLX-001 40 mg QD Oral.

| | | | | | | AUCₗₐₛₜ or | | | |
|---|---|---|---|---|---|---|---|---|---|
| | | Dose | Half-life | Tₘₐₓ | Cₘₐₓ | AUCₜₐᵤ, | AUC_{inf} | AUCₑₓₜᵣₐₚ | Concomitant |
| Day | Patient | (mg) | (h) | (h) | (ng/mL) | (ng*h/mL) | (ng*h/mL) | (%) | Pantoprazole |
| 1 | 115-002-006 | 140 | 7.91 | 0.5 | 2710 | 21234 | 24165 | 5.48 | No |
| 1 | 115-024-013 | 140 | 5.86 | 1 | 2190 | 10879 | 11510 | 12.5 | Yes |
| 1 | 115-024-005 | 140 | 7.56 | 1 | 406 | 3634 | 4155 | 12.1 | Yes |
| 15 | 115-002-006 | 140 | 16.4 | 2 | 2200 | 25987 | 41870 | 22.7 | No |
| 15 | 115-024-013 | 140 | 10.1 | 1 | 1510 | 11043 | 14292 | 39.9 | Yes |
| 15 | 115-024-005 | 140 | 17.4 | 2 | 295 | 4318 | 7185 | 37.9 | Yes |

Note:
The AUCextrap highlighted in red and underlined if >15% of AUCinf is extrapolated.

It is shown herein, there is: greater exposure of PCLX-001 than anticipated from animal data (longer half-life drives this, in large part) and rapid oral absorption of PCLX-001.

It is also shown malabsorption/small bowel resection delays absorption and reduces exposure.

It is also shown interaction with proton pump inhibitors, such as pantoprazole (low gastric acidity) reduces oral absorption

It is also shown that trough drug levels (i.e., pre-dose levels) can be achieved that markedly exceed the IC50 and IC90s of cultured cancer cell lines treated with PCLX-001 in tissue culture, suggesting therapeutic drug levels and exposures can be achieved in humans.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A method for treating cancer in a subject in need thereof, comprising, administering an oral dosage form of PCLX-001, wherein said oral dosage comprises 20 mg PCLX-001 per day, 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 mg PCLX-001 per day.

2. The method of claim 1, wherein said oral dosage provides an effect comprising a T1/2 of about 5 hours to about 18 hours.

3. The method of claim 1 or 2, wherein said oral dosage provides an effect comprising a T1/2 of about 5.5 hours to about 14 hours.

4. A method for treating cancer in a subject in need thereof, comprising, administering an oral dosage form of PCLX-001, wherein said oral dosage comprises 20 mg PCLX-001 per day.

5. The method of claim 4, wherein said oral dosage provides an effect comprising a T1/2 of about 6 hours to about 9 hours.

6. The method of claim 4 or 5, wherein said oral dosage provides an effect comprising a T1/2 of about 6.9 hours to about to about 8.9 hours.

7. A method for treating cancer in a subject in need thereof, comprising, administering an oral dosage form of PCLX-001, wherein said oral dosage comprises 40 mg PCLX-001 per day.

8. The method of claim 7, wherein said oral dosage provides an effect comprising a T1/2 of about 5 hours to about 11 hours.

9. The method of claim 7 or 8, wherein said oral dosage provides an effect comprising a T1/2 of about 5.5 hours to about 10.5 hours.

10. A method for treating cancer in a subject in need thereof, comprising, administering an oral dosage form of PCLX-001, wherein said oral dosage comprises 70 mg PCLX-001 per day.

11. The method of claim 10, wherein said oral dosage provides an effect comprising a T1/2 of about 9 hours.

12. The method of claim 10 or 11, wherein said oral dosage provides an effect comprising a T1/2 of about 9.4 hours.

13. A method for treating cancer in a subject in need thereof, comprising, administering an oral dosage form of PCLX-001, wherein said oral dosage comprises 100 mg PCLX-001 per day.

14. The method of claim 13, wherein said oral dosage provides an effect comprising a T1/2 of about 7 hours to about 8 hours.

15. The method of claim 13 or 14, wherein said oral dosage provides an effect comprising a T1/2 of about 7.2 hours to about 7.5 hours.

16. A method for treating cancer in a subject in need thereof, comprising, administering an oral dosage form of PCLX-001, wherein said oral dosage comprises 140 mg PCLX-001 per day.

17. The method of claim 13, wherein said oral dosage provides an effect comprising a T1/2 of about 6 hours to about 14 hours.

18. The method of claim 16 or 17, wherein said oral dosage provides an effect comprising a T1/2 of about 6.7 hours to about 13.8 hours.

19. The method of any one of claims 1 to 18, wherein the cancer is Diffuse Large B Cell Lymphoma (DLBCL).

20. The method of claim 19, wherein said DLBC is refractory DLBC.

21. The method of any one of claims 1 to 18, wherein the cancer is lymphoma, leukemia, myeloma, breast cancer, small cell lung cancer, non-small cell lung cancer cells; melanoma, adenocarcinoma, pancreas cancer, bladder cancer, ovarian cancer, brain cancer, colon cancer, neuroblastoma, carcinoma, endometrial cancer, sarcoma, thyroid cancer, fibrosarcoma, oral cancer, tongue cancer, nasopharynx cancer, larynx cancer, esophageal cancer, germ cell cancer, stomach cancer, hepatocellular cancer, biliary tract cancer, small intestinal cancer, testis cancer, epidermoid cancer, cervical cancer, kidney cancer, prostate cancer, or pharynx cancer.

22. The method of any one of claims 1 to 21, wherein said subject is a human.

23. Use of an oral dosage form of PCLX-001 for treating cancer in a subject in need thereof, wherein said oral dosage comprises 20 mg PCLX-001 per day, 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 mg PCLX-001 per day.

24. Use of an oral dosage form of PCLX-001 in the manufacture of a medicament for treating cancer in a subject in need thereof, wherein said oral dosage comprises 20 mg PCLX-001 per day, 40 mg PCLX-001 per day, 70 mg PCLX-001 per day, 100 mg PCLX-001 per day, 140 mg PCLX-001 per day, 210 mg PCLX-001 per day, 280 mg PCLX-001 per day, 350 mg PCLX-001 per day, or 420 mg PCLX-001 per day.

25. The use of claim 23 or 24, wherein said oral dosage provides an effect comprising a T1/2 of about 5 hours to about 18 hours.

26. The use of any one of claims 23 to 25, wherein said oral dosage provides an effect comprising a T1/2 of about 5.5 hours to about 14 hours.

27. Use of an oral dosage form of PCLX-001 for treating cancer in a subject in need thereof, wherein said oral dosage comprises 20 mg PCLX-001 per day.

28. Use of an oral dosage form of PCLX-001 in the manufacture of a medicament for treating cancer in a subject in need thereof, wherein said oral dosage comprises 20 mg PCLX-001 per day.

29. The use of claim 27 or 28, wherein said oral dosage provides an effect comprising a T1/2 of about 6 hours to about 9 hours.

30. The use of any one of claims 27 to 29, wherein said oral dosage provides an effect comprising a T1/2 of about 6.9 hours to about to about 8.9 hours.

31. Use of an oral dosage form of PCLX-001 for treating cancer in a subject in need thereof, wherein said oral dosage comprises 40 mg PCLX-001 per day.

32. Use of an oral dosage form of PCLX-001 in the manufacture of a medicament for treating cancer in a subject in need thereof, wherein said oral dosage comprises 40 mg PCLX-001 per day.

33. The use of claim 31 or 32, wherein said oral dosage provides an effect comprising a T1/2 of about 5 hours to about 11 hours.

34. The use of any one of claims 31 to 33, wherein said oral dosage provides an effect comprising a T1/2 of about 5.5 hours to about 10.5 hours.

35. Use of an oral dosage form of PCLX-001 for treating cancer in a subject in need thereof, wherein said oral dosage comprises 70 mg PCLX-001 per day.

36. Use of an oral dosage form of PCLX-001 in the manufacture of a medicament for treating cancer in a subject in need thereof, wherein said oral dosage comprises 70 mg PCLX-001 per day.

37. The use of claim 35 or 36, wherein said oral dosage provides an effect comprising a T1/2 of about 9 hours.

38. The use of any one of claims 35 to 37, wherein said oral dosage provides an effect comprising a T1/2 of about 9.4 hours.

39. Use of an oral dosage form of PCLX-001 for treating cancer in a subject in need thereof, wherein said oral dosage comprises 100 mg PCLX-001 per day.

40. Use of an oral dosage form of PCLX-001 in the manufacture of a medicament for treating cancer in a subject in need thereof, wherein said oral dosage comprises 100 mg PCLX-001 per day.

41. The use of claim 39 or 40, wherein said oral dosage provides an effect comprising a T1/2 of about 7 hours to about 8 hours.

42. The use of any one of claim 39 or 41, wherein said oral dosage provides an effect comprising a T1/2 of about 7.2 hours to about 7.5 hours.

43. Use of an oral dosage form of PCLX-001 for treating cancer in a subject in need thereof, wherein said oral dosage comprises 140 mg PCLX-001 per day.

44. Use of an oral dosage form of PCLX-001 in the manufacture of a medicament for treating cancer in a subject in need thereof, wherein said oral dosage comprises 140 mg PCLX-001 per day.

45. The use of claim 43 or 44, wherein said oral dosage provides an effect comprising a T1/2 of about 6 hours to about 14 hours.

46. The use of any one of claims 43 to 45, wherein said oral dosage provides an effect comprising a T1/2 of about 6.7 hours to about 13.8 hours.

47. The use of any one of claims 23 to 46, wherein the cancer is Diffuse Large B Cell Lymphoma (DLBCL).

48. The method of claim 47, wherein said DLBC is refractory DLBC.

49. The use of any one of claims 23 to 46, wherein the cancer is lymphoma, leukemia, myeloma, breast cancer, small cell lung cancer, non-small cell lung cancer cells; melanoma, adenocarcinoma, pancreas cancer, bladder cancer, ovarian cancer, brain cancer, colon cancer, neuroblastoma, carcinoma, endometrial cancer, sarcoma, thyroid cancer, fibrosarcoma, oral cancer, tongue cancer, nasopharynx cancer, larynx cancer, esophageal cancer, germ cell cancer, stomach cancer, hepatocellular cancer, biliary tract cancer, small intestinal cancer, testis cancer, epidermoid cancer, cervical cancer, kidney cancer, prostate cancer, or pharynx cancer.

50. The use of any one of claims 23 to 49, wherein said subject is a human.
